SEQUENCE LISTING (9 sequences)

SEQ ID NO: 1 - protein, 320 aa, Artificial Sequence
  ProDerp1
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 2 - DNA, 22 bp, Artificial Sequence
  Primer
gtatctctcgagaaaagagagg

SEQ ID NO: 3 - DNA, 31 bp, Artificial Sequence
  Primer
gcggccgcgattagagaatgacaacatatgg

SEQ ID NO: 4 - DNA, 31 bp, Artificial Sequence
  Primer
ggaggctgtggttcagcttgggctttctctg

SEQ ID NO: 5 - DNA, 31 bp, Artificial Sequence
  Primer
cagagaaagcccaagctgaaccacagcctcc

SEQ ID NO: 6 - DNA, 40 bp, Artificial Sequence
  Primer
gaatacaaaaaagccttccagaaaagttatgctaccttcg

SEQ ID NO: 7 - DNA, 40 bp, Artificial Sequence
  Primer
cgaaggtagcataacttttctggaaggcttttttgtattc

SEQ ID NO: 8 - DNA, 38 bp, Artificial Sequence
  Primer
gcttatttggcttaccgtcagcaatcattggatcttgc

SEQ ID NO: 9 - DNA, 38 bp, Artificial Sequence
  Primer
gcaagatccaatgattgctgacggtaagccaaataagc

(19) United States
(12) Patent Application Publication
(10) Pub. No.: US 2021/0261604 A1
(43) Pub. Date: Aug. 26, 2021

(54) COMPOSITIONS FOR ACTIVE IMMUNOTHERAPY

(71) Applicants: VIB VZW, Gent (BE); UNIVERSITEIT GENT, Gent (BE)

(72) Inventors: Nico Callewaert, Nevele (BE); Charlot De Wachter, Gent (BE)

(21) Appl. No.: 17/078,883

(22) PCT Filed: Apr. 26, 2019
(86) PCT No.: PCT/EP2019/060747

§ 371 Date: Oct. 23, 2020

(30) Foreign Application Priority Data
Apr. 26, 2018 (GB) 1806819.7

(51) Int. Cl. C07K 1/12; A61K 45/06; C07K 14/435

(52) U.S. Cl. CPC C07K 1/122; C07K 14/43531; A61K 45/06

(57) ABSTRACT

The present invention provides to novel prophylactic and therapeutic formulations being effective in the prevention and/or the reduction of allergenic responses to specific allergens. In particular the invention provides compositions comprising deglycosylated allergens which allergens are normally glycosylated in their natural environment. Further this invention further relates to hypoallergenic recombinant deglycosylated derivatives of the major protein allergen from Dermatophagoides pteronyssinus, allergen proDerp1. Even more particularly the invention further provides hypoallergenic recombinant deglycosylated derivatives of proDerp1 of which catalytic cysteine 132 is mutated.

### CROSS-REFERENCE TO RELATED APPLICATIONS

This application is a national phase entry under 35 U.S.C. § 371 of International Patent Application PCT/EP2019/060747, filed Apr. 26, 2019, designating the United States of America and published in English as International Patent Publication WO 2019/207109 on Oct. 31, 2019, which claims the benefit under Article 8 of the Patent Cooperation Treaty to United Kingdom Patent Application Serial No. 1806819.7, filed Apr. 26, 2018, the entireties of which are hereby incorporated by reference.

### FIELD OF THE INVENTION

The present invention relates to novel prophylactic and therapeutic formulations being effective in the prevention and/or the reduction of allergenic responses to specific allergens. In particular the invention relates to compositions comprising deglycosylated allergens which allergens are normally glycosylated in their natural environment. Further this invention further relates to hypoallergenic recombinant derivatives of the major protein allergen from Dermatophagoides pteronyssinus, allergen proDerp1.

### INTRODUCTION TO THE INVENTION

Allergic responses in humans are common, and may be triggered by a variety of allergens. Allergic individuals are sensitised to allergens, and are characterised by the presence of high levels of allergen specific IgE in the serum, and possess allergen specific T-cell populations which produce Th2-type cytokines (IL-4, IL-5, and IL-13). Binding of IgE, in the presence of allergen, to FcεRI receptors present on the surface of mastocytes and basophils, leads to the rapid degranulation of the cells and the subsequent release of histamine, and other preformed and neoformed mediators of the inflammatory reaction. In addition to this, the stimulation of the T-cell recall response results in the production of IL-4 and IL-13, together cooperating to switch B-cell responses further towards allergen specific IgE production. Type I allergic diseases mediated by IgE against allergens such as bronchial asthma, atopic dermatitis and perennial rhinitis affect more than 20% of the world's population.

In particular asthma is a continuously growing healthcare problem, with more than 300 million of people affected worldwide. In about 60% of all asthmatic patients, allergy is the underlying causative factor with more than 50% of the allergic asthma attacks evoked by house dust mite (HDM) allergens. Glucocorticosteroids, antihistamines and bronchodilators are amongst the most used pharmacotherapeutics to relieve asthmatic symptoms. However, these drugs lack a prolonged effect requiring a daily intake during allergy season. Additionally, long-term usage of glucocorticosteroids has been shown to induce side effects as well as drug resistance. So far, the only therapy with the potential to cure the disease is allergen-specific immunotherapy (AIT), in which the repeated administration of a gradually increasing dose of allergen extract aims to desensitize patients and in that way prevent future allergic reactions. Although this treatment is the only therapy with long-lasting potential, its clinical practice remains limited to severe allergic asthma patients partly due to the high risk of local and systemic side effects, together with the long duration of the treatment, up to 3-5 years. A major trigger of side effects is the batch-to-batch and manufacturer-to-manufacturer variation of the allergen content between allergen extracts. These allergens may cross-link IgE-FcεR complexes on effector cells and trigger allergic reactions, including anaphylactic shock. This variation in allergen content, as well as contamination of the allergen extracts with additional antigens or macromolecules, causes differences in allergenicity and immunogenicity. As there is clearly ample room for improvement, AIT is an extensively studied field in which the ideal composition of the immunotherapeutic, together with the route of administration and helper factors, such as adjuvants, is investigated to increase its safety profile and efficacy.

The use of recombinant allergens allows for the production of highly standardized and reproducible immunotherapeutics, offering a solution to the high variability of allergen extracts. In the present invention, we focused on house dust mite (HDM)-induced asthma and the use of Der p 1, a major HDM allergen, for the optimization of AIT. The use of recombinant Der p 1, however, retains the intrinsic allergenic properties of the allergen caused by both its protease activity and conformation, the latter triggering IgE-dependent allergic reactions. To avoid this, we produced a hypoallergenic form of Der p 1, id est ProDerp1 in the yeast P. pastoris. In ProDerp1 the pro-peptide is still present and masks conformational IgE-binding epitopes (Takai T. et al (2005) J. Allergy Clin. Immunol. 115, 555-563). In addition, the pro-peptide shields the enzymatic active site, further reducing the allergenicity (Walgraffe D et al (2009) J. Allergy Clin. Immunol. 123, 1150-1156). We explored whether different glycosylated forms of ProDer p 1 had an impact on the efficacy of the molecule as an AIT immunotherapeutic since it has been shown in the art that high-mannose N-glycans are associated with the allergenicity of proteins (Al-Ghouleh A et al (2012) PLoS ONE 7, e33929), while antibody-based targeting of antigens to the macrophage-galactose C-type lectin (MGL) induced IL-10-producing suppressive CD4⁺ T cells (Li D et al (2012) J. Exp. Med. 209, 109-121). Therefore, we produced a variety of ProDer p 1 glycoforms modified with specific N-glycan structures designed to target specific lectins present on antigen-presenting cells, in particular mannose-binding lectins and MGL, in order to investigate the influence of lectin-based internalization on the triggered immune response. It was unexpectedly found that the influence of the differently N-glycosylated ProDer p 1 forms on the protection against HDM-induced asthma was of little relevance. Instead we showed that deglycosylated ProDer p 1 forms performed much better in inducing tolerance in an HDM-driven asthma murine model (for the murine model see Debeuf N. et al (2016) Curr. Protoc. Mouse Biol. 6, 169-184). This is a striking observation since a mutant ProDerp1 lacking N-glycosylation acceptor sites and having a cysteine 132 mutation (C132V) is less potent in inducing tolerance (Burtin D. et al (2009) Clinical and Experimental Allergy 39, 760). Thus in contrast to what is described in the art we could not observe any consistent difference in tolerance induction between treatment with oligo-mannose, GalNAc₃GlcNAc₃Man₃GlcNAc₂ or GalNAc-β1,4-GlcNAc (LDN) N-glycan modified ProDerp1 forms. Importantly we could also not confirm the induction of a regulatory immune phenotype by MGL-targeting as was previously described by Li and colleagues.

In addition, we showed that the most promising protective effects against HDM-induced asthma were obtained with enzymatically deglycosylated ProDerp1 which had the catalytic Cysteine on position 132 inactivated via iodoalkylation. The present invention has implications for generating hypoallergenic forms of antigens which are naturally glycosylated. We surprisingly show that enzymatic deglycosylation of naturally glycosylated allergens leads to hypoallergenic versions of said allergens.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1D: Prophylactic treatment with ProDer p 1 (C132A) glycoforms in a HDM-induced asthma mouse model. FIG. 1A. Protein analysis on a Coomassie-stained SDS-PAGE gel of the engineered IAA-modified ProDer p 1 and ProDer p 1 C132A forms included in this in vivo experiment. M5: IAA-modified Man₅GlcNAc₂ ProDer p 1, M5 C132A: Man₅GlcNAc₂ ProDer p 1 C132A, LDN C132A: LDN ProDer p 1 C132A, GalNAc3: IAA-modified GalNAc₃GlcNAc₃Man₃GlcNAc₂ ProDer p 1, DG: IAA-modified deglycosylated ProDer p 1. FIG. 1B. N-glycosylation profile of purified IAA-modified ProDer p 1 and ProDer p 1 C132A forms analyzed with CE-LIF. Malto-oligosaccharide standard with single glucose units corresponding to the peak-to-peak shift (Panel 1). N-glycosylation profiles of purified IAA-modified Man₅GlcNAc₂ ProDer p 1 (Panel 2), Man₅GlcNAc₂ ProDer p 1 C132A (Panel 3), IAA-modified deglycosylated ProDer p 1 (Panel 4), LDN ProDer p 1 C132A (Panel 6), IAA-modified GalNAc₃GlcNAc₃Man₃GlcNAc₂ ProDer p 1 (Panel 7) and PBS as a negative control (Panel 8). RNase B N-glycan standard with a typical profile consisting of Man₅₋₉GlcNAc₂ N-glycans (M5-M9) (Panel 5). FIG. 1C. Time scheme of the experimental work-flow followed during prophylactic treatment in a HDM-induced asthma mouse model. T₁₋₄: treatments 1-4 on days-14, -11, -7 and -1, S: sensitization on day 0, C: challenge on days 7-11, and A: analysis on day 14. FIG. 1D. BAL fluid was analyzed for eosinophils, neutrophils, T cells, B cells, macrophages and dendritic cells. Each data point represents 1 mouse, black and grey data points were obtained on different days. In addition, the mean of all data points is shown. *p<0.05; **p<0.01; ***p<0.001; ****p<0.0001. Deglycosylated: IAA-modified deglycosylated ProDer p 1, GalNAc3: IAA-modified GalNAc₃GlcNAc₃Man₃GlcNAc₂ ProDer p 1, Man5: IAA-modified Man₅GlcNAc₂ ProDer p 1, Man5 C132A: Man₅GlcNAc₂ ProDer p 1 C132A, LDN: LDN ProDer p 1 C132A.

FIGS. 2A-2D: Prophylactic treatment in a HDM-induced asthma C57BL/6J mouse model: FIG. 2A. Time scheme of the experimental work-flow followed during prophylactic treatment in a house dust mite-induced asthma mouse model. T₁₋₄: i.n. treatments 1-4 (50 μg ProDer p 1 variant/40 μl PBS) on days-14, -11, -7 and -1, S: i.t. sensitization (1 μg HDM extract/80 μl PBS) on day 0, C: i.n. challenge (10 μg HDM extract/40 μl PBS) on days 7-11, and A: analysis on day 14. FIG. 2B. The number of eosinophils, neutrophils, T cells, B cells, dendritic cells and macrophages present in the BAL fluid were quantified using flow cytometry. Each data point represents an individual mouse, and the mean of all data points is shown. FIG. 2C. Lung-draining MLNs were isolated and restimulated with HDM extract. Growth medium was collected and analyzed for expression of IL-13, IL-5, IL-10, IL-17A and IFN-γ using ELISA. Graphs show mean+SEM, n=4-8. FIG. 2D. HDM-specific IgE blood levels were measured using ELISA. Graph shows mean+SEM, n=8. *p<0.05, **p<0.01; ***p<0.001. Man5 C132A: Man₅GlcNAc₂ ProDer p 1 C132A, NG C132A: non-glycosylated ProDer p 1 C132A/N34Q/N150Q, DG: IAA-modified deglycosylated ProDerp1.

FIGS. 3A-3B: Prophylactic treatment in a long-term HDM-induced asthma C57BL/6J mouse model: FIG. 3A. Time scheme of the experimental work-flow followed during prophylactic treatment in a long-term house dust mite-induced asthma mouse model. T₁₋₄: i.n. treatments 1-4 (50 μg ProDer p 1 variant/40 μl PBS) on days-14, -11, -7 and -1, S: i.t. sensitization (1 μg HDM extract/80 μl PBS) on day 0, C: i.n. challenge (10 μg HDM extract/40 μl PBS) on days 48-52, A: analysis on day 55. FIG. 3B. The number of eosinophils, neutrophils, T cells, B cells, dendritic cells and macrophages present in the BAL fluid were quantified using flow cytometry. Each data point represents an individual mouse, and the mean of all data points is shown. No significant differences were obtained. Man5 C132A: Man₅GlcNAc₂ ProDer p 1 C132A, NG C132A: non-glycosylated ProDer p 1 C132A/N34Q/N150Q, DG: IAA-modified deglycosylated ProDerp1.

FIGS. 4A-4B: The minimal required number of IAA-modified DG ProDer p 1 treatments to obtain a protective effect. FIG. 4A. Time scheme of the experimental work-flow followed during prophylactic treatment in a HDM-induced asthma C57BL/6J mouse model. T₁₋₄: i.n. treatments (50 μg/40 μl PBS) on days-14, -11, -7 and -1, S: i.t. sensitization (1 μg HDM extract/80 μl PBS) on day 0, C: i.n. challenge (10 μg HDM extract/40 μl PBS) on days 7-11, and A: analysis on day 14. FIG. 4B. The number of eosinophils, neutrophils, T cells, B cells, macrophages and dendritic cells present in the BAL fluid were quantified using flow cytometry. Each data point represents an individual mouse, and the mean of all data points is shown. *p<0.05, **p<0.01.

FIGS. 5A-5B: Prophylactic treatment in a HDM-induced asthma Balb/c mouse model: FIG. 5A. Time scheme of the experimental work-flow followed during prophylactic treatment in a house dust mite-induced asthma mouse model. T₁₋₄: i.n. treatments 1-4 (50 μg ProDer p 1 variant/40 μl PBS) on days-14, -11, -7 and -1, S: i.t. sensitization (1 μg HDM extract/80 μl PBS) on day 0, C: i.n. challenge (10 μg HDM extract/40 μl PBS) on days 7-11, and A: analysis on day 14. FIG. 5B. The number of eosinophils, neutrophils, T cells, B cells, dendritic cells and macrophages present in the BAL fluid were quantified using flow cytometry. Each data point represents an individual mouse, and the mean of all data points is shown. *p<0.05. Man5: IAA-modified Man₅GlcNAc₂ ProDer p 1, DG: IAA-modified deglycosylated ProDer p 1.

FIG. 6: Antigen uptake of labeled ProDer p 1 variants by macrophages and dendritic cells (DCs) of BAL fluid: Cells obtained from BAL fluid were stimulated with 20 μg/ml of a labeled ProDer p 1 form and harvested 0, 3 or 6 hours post stimulus. Antigen uptake followed over time was represented either as the percentage of dendritic cells or macrophages which have taken up the antigen (left), or by measuring the mean fluorescence induction (MFI) of Ag-AF488 displayed as AMFI compared to the unstimulated population (right). M5: Man₅GlcNAc₂, DG: deglycosylated.

FIG. 7A-7C: SDS-PAGE analysis of purified DG, NG and Man₅GlcNAc₂ IAA-modified ProDer p 1 and ProDer p 1 C132A. FIG. 7A. Glycoproteins were visualized using the PAS reagent method. FIG. 7B. Proteins were visualized using Coomassie-staining. FIG. 7C. Proteins were visualized using rabbit anti-Der p 1 primary antibody, which is in turn detected by a goat anti-rabbit antibody coupled to Dylight® 800 and visualized by a LI-COR Odyssey system. Lane 1: IAA-modified DG ProDer p 1, lane 2: IAA-modified Man₅GlcNAc₂ ProDer p 1, lane 3: IAA-modified NG ProDer p 1 N34Q/N150Q, lane 4: DG ProDer p 1 C132A, lane 5: Man₅GlcNAc₂ ProDer p 1 C132A, lane 6: NG ProDer p 1 C132A/N34Q/N150Q, lane 7: horseradish peroxidase as positive control in the Glycoprotein Staining kit, lane 8: soybean trypsin inhibitor as negative control in the Glycoprotein Staining kit.

FIG. 8A-8C: Thermofluor assay and circular dichroism measurements. FIG. 8A. The graph shows the melting curves of IAA-modified ProDer p 1 N34Q/N150Q, ProDer p 1 C132A/N34Q/N150Q, IAA-modified DG ProDer p 1 and PBS obtained from a thermofluor assay at a protein concentration of 225 ng/μl. FIG. 8B. Circular dichroism spectra of IAA-modified ProDer p 1 N34Q/N150Q, ProDer p 1 C132A/N34Q/N150Q and IAA-modified DG ProDer p 1 with most qualitative data in the range of 200-260 nm (high tension voltage<600 V). FIG. 8C. Prediction of the secondary structure contents based on the BeStSel algorithm (bestsel.elte.hu) applied to 200-250 nm range.

FIG. 9: SEC-MALLS analysis of IAA-modified Man₅GlcNAc₂ ProDer p 1, IAA-modified DG ProDer p 1, IAA-modified NG ProDer p 1 N34Q/N150Q and NG ProDer p 1 C132A/N34Q/N150Q. The SEC-MALLS analysis was performed on a Superdex 200 Increase (GE Healthcare), in-line with an online UV-detector (Shimadzu), a light scattering detector (Wyatt) and a refractive index detector (Wyatt). SEC elution profiles of ProDer p 1 variants at a concentration of 0.5 mg/ml are shown. The three lines represent the calculated molecular weight for the glycan, the protein and the total molecular weight (=glycan+protein).

FIGS. 10A-10B: Schematic overview of the different ProDer p 1 forms generated in the present invention. 1) Man₅GlcNAc₂ ProDer p 1 is produced in the GlycoSwitchM5® P. pastoris strain and modified with IAA before purification (indicated as AA). A type 1 clipping event occurs either during production or during purification. 2) Man₅GlcNAc₂ ProDer p 1 is produced in the GlycoSwitchM5® P. pastoris strain and modified with IAA before purification (indicated as AA). A type 1 clipping event occurs either during production or during purification. After purification, the Man₅GlcNAc₂ ProDer p 1 form is deglycosylated in vitro using PNGase F in non-denatured conditions, resulting in the loss of N-glycans and in the deamination of N to D. This form induces the strongest protective effect by prophylactic treatment in a HDM-driven allergic asthma mouse model. 3) ProDer p 1 N34Q/N150Q is produced in the GS115 P. pastoris strain and modified with IAA before purification. A type 2 clipping event occurs either during production of during purification. 4) Man₅GlcNAc₂ ProDer p 1 C132A is produced in the GlycoSwitchM5® P. pastoris strain. 5) Man₅GlcNAc₂ ProDer p 1 C132A is produced in the GlycoSwitchM5® P. pastoris strain. Post-purification deglycosylation of the protein in non-denatured conditions with PNGase F was incomplete. 6) ProDer p 1 C132A/N34Q/N150Q is produced in the GS115 P. pastoris strain.

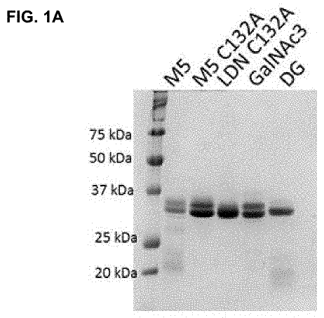

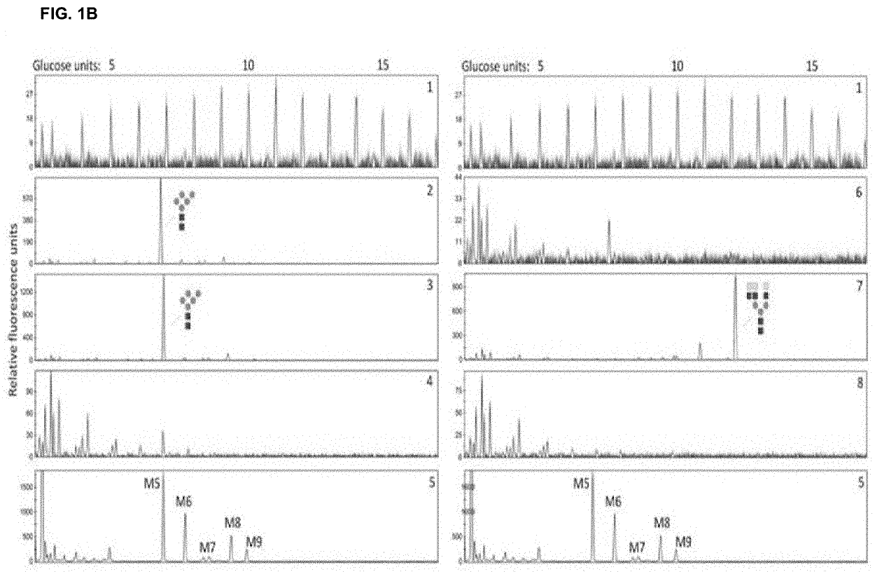

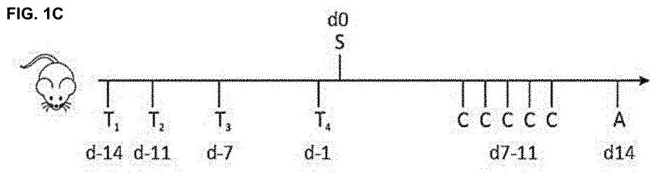

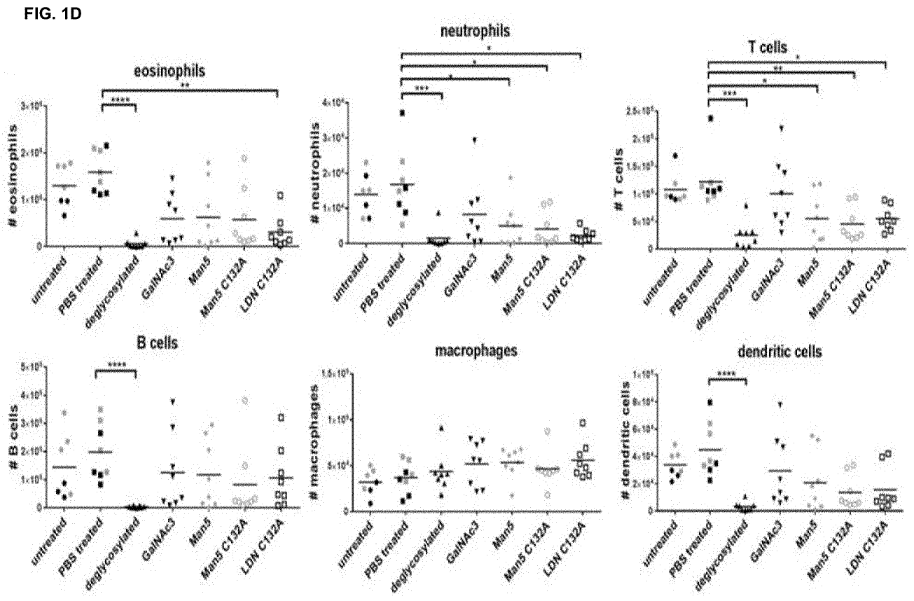

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

As used herein, the term “nucleotide sequence” refers to a polymeric form of nucleotides of any length, either deoxyribonucleotides or ribonucleotides, or analogs thereof. Nucleotide sequences may have any three-dimensional structure, and may perform any function, known or unknown. Non-limiting examples of nucleotide sequences include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleotide sequence may be linear or circular.

As used herein, the term “polypeptide” refers to a polymeric form of amino acids of any length, which can include coded and non-coded amino acids, chemically or biochemically modified or derivatized amino acids, and polypeptides having modified peptide backbones. Polypeptide sequences can be depicted with the single-letter (or one letter) amino acid code or the three letter amino acid code as depicted here below:

| Amino acid | Three letter code | One letter code |
|---|---|---|
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |

The term “Glycosylation acceptor site” refers to a position within the allergen (e.g. proDerp1), which can be N- or O-glycosylated. N-linked glycans are typically attached to Asparagine (Asn), while O-linked glycans are commonly linked to the hydroxyl oxygen of serine, threonine, tyrosine, hydroxylysine, or hydroxyproline side-chains.

The term “expression vector”, as used herein, includes any vector known to the skilled person, including plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Expression vectors generally contain a desired coding sequence and appropriate promoter sequences necessary for the expression of the operably linked coding sequence in a particular host organism (e.g. higher eukaryotes, lower eukaryotes, prokaryotes).

Typically, a vector comprises a nucleotide sequence in which an expressible promoter or regulatory nucleotide sequence is operatively linked to, or associated with, a nucleotide sequence or DNA region that codes for an mRNA, such that the regulatory nucleotide sequence is able to regulate transcription or expression of the associated nucleotide sequence. Typically, a regulatory nucleotide sequence or promoter of the vector is not operatively linked to the associated nucleotide sequence as found in nature, hence is heterologous to the coding sequence of the DNA region operably linked to. The term “operatively” or “operably” “linked” as used herein refers to a functional linkage between the expressible promoter sequence and the DNA region or gene of interest, such that the promoter sequence is able to initiate transcription of the gene of interest, and refers to a functional linkage between the gene of interest and the transcription terminating sequence to assure adequate termination of transcription in eukaryotic cells. An “inducible promoter” refers to a promoter that can be switched ‘on’ or ‘off’ (thereby regulating gene transcription) in response to external stimuli such as, but not limited to, temperature, pH, certain nutrients, specific cellular signals, et cetera. It is used to distinguish between a “constitutive promoter”, by which a promoter is meant that is continuously active.

A “glycan” as used herein generally refers to glycosidically linked monosaccharides, oligosaccharides and polysaccharides. Hence, carbohydrate portions of a glycoconjugate, such as a glycoprotein, glycolipid, or a proteoglycan are referred to herein as a “glycan”. Glycans can be homo- or heteropolymers of monosaccharide residues, and can be linear or branched. N-linked glycans may be composed of GalNAc, Galactose, neuraminic acid, N-acetylglucosamine, Fucose, Mannose, and other monosaccharides, as also exemplified further herein.

In eukaryotes, O-linked glycans are assembled one sugar at a time on a serine or threonine residue of a peptide chain in the Golgi apparatus. Unlike N-linked glycans, there are no known consensus sequences but the position of a proline residue at either −1 or +3 relative to the serine or threonine is favourable for O-linked glycosylation.

“Complex N-glycans” as used in the application refers to structures with typically one, two or more (e.g. up to six) outer branches, most often linked to an inner core structure Man3GlcNAc2. The term “complex N-glycans” is well known to the skilled person and defined in literature. For instance, a complex N-glycan may have at least one branch, or at least two, of alternating GlcNAc and optionally also Galactose (Gal) residues that may terminate in a variety of oligosaccharides but typically will not terminate with a Mannose residue.

A “higher eukaryotic cell” as used herein refers to eukaryotic cells that are not cells from unicellular organisms. In other words, a higher eukaryotic cell is a cell from (or derived from, in case of cell cultures) a multicellular eukaryote such as a human cell line or another mammalian cell line (e.g. a CHO cell line). Typically, the higher eukaryotic cells will not be fungal cells. Particularly, the term generally refers to mammalian cells, human cell lines and insect cell lines. More particularly, the term refers to vertebrate cells, even more particularly to mammalian cells or human cells. The higher eukaryotic cells as described herein will typically be part of a cell culture (e.g. a cell line, such as a HEK or CHO cell line), although this is not always strictly required (e.g. in case of plant cells, the plant itself can be used to produce a recombinant protein).

By “lower eukaryotic cell” a filamentous fungus cell or a yeast cell is meant. Yeast cells can be from the species Saccharomyces (e.g. Saccharomyces cerevisiae), Hansenula (e.g. Hansenula polymorpha), Arxula (e.g. Arxula adeninivorans), Yarrowia (e.g. Yarrowia lipolytica), Kluyveromyces (e.g. Kluyveromyces lactis), or Komagataella phaffii (Kurtzman, C. P. (2009) J Ind Microbiol Biotechnol. 36(11) which was previously named and better known under the old nomenclature as Pichia pastoris and also further used herein. According to a specific embodiment, the lower eukaryotic cells are Pichia cells, and in a most particular embodiment Pichia pastoris cells. In specific embodiments the filamentous fungus cell is Myceliopthora thermophila (also known as C1 by the company Dyadic), Aspergillus species (e.g. Aspergillus nidulans, Aspergillus niger, Aspergillus oryzae, Aspergillus japonicus), Fusarium species (e.g. Fusarium venenatum), Hypocrea and Trichoderma species (e.g. Trichoderma reesei).

“Prokaryotic cells” typically refer to non-pathogenic prokaryotes like bacterial cells such as for example E. coli, Lactococcus and Bacillus species.

In the majority of allergic asthma patients, attacks are provoked by house dust mite allergens, which originate from one of the two principal mite species Dermatophagoides pteronyssinus and Dermatophagoides farinae. More than 80% of the patients have IgE antibodies against the excreted Group 1 allergen of one of both mite species, Der p 1 (for D. pteronyssinus) or Der f 1 (for D. farinae). Both allergens are cysteine proteases and share 81% of sequence identity, causing IgE cross-reactivity. Especially for Derp1, the proteolytic activity has been shown to contribute to allergenicity by disruption of the tight junctions in the lung epithelium, hereby increasing the accessibility to dendritic cells and the formation of IgE-FcεRI complexes on mast cells and basophils.

The amino acid sequence of ProDerp1 is depicted in SEQ ID NO: 1:

```
1 10 20 30 40
MKIVLAIASLLALSAVYA RPSSIKTFEEYKKAFNKSYATFED
      signal peptide
              50 60 70 80
EEAARKNFLESVKYVQSNGGAINHLSDLSLDEFKNRFLMSAEAFE
pro-peptide
  90 100 110 120 130
HLKTQFDLNAE TNACSINGNAPAEIDLRQMRTVTPIRMQGGCGSC
            140 150 160
WAFSGVAATESAYLAYRNQSLDLAEQELVDCASQHGC
catalytic domain
170 180 190 200 210
HGDTIPRGIEYIQHNGVVQESYYRYVAREQSCRRPNAQRFGISNYCQIY
220 230 240 250
PPNANKIREALAQTHSAIAVIIGIKDLDAFRHYDGRT
      260 270 280 290
IIQRDNGYQPNYHAVNIVGYSNAQGVDYWIVRNSWDTNWGDNG
300 310 320
YGYFAANIDLMMIEEYPYVVIL
```

SEQ ID NO: 1 depicts the protein sequence of ProDerp1: Amino acids 1-18 represent the natural signal peptide (underlined), residues 19-98 represent the pro-sequence (underlined) and residues 99-320 represent the catalytic domain (underlined) of the allergen. The pro-containing allergen contains two putative N-glycosylation sites (highlighted in grey), and the cysteine residue (C132) required for the allergen's protease activity is indicated in dark grey.

Derp1 is produced as an immature protein (ProDerp1) composed of a 25 kDa catalytic domain of 222 amino acids preceded by a 9 kDa N-terminal pro-peptide of 80 amino acids (see SEQ ID NO: 1). As suggested for other proteases, this pro-peptide may act as a scaffold to guide proper folding of Derp1, after which the pro-peptide is processed to obtain the active enzyme. The immature ProDer p 1 form has been shown to have reduced enzymatic activity as the pro-peptide interacts with the active site cleft and adjacent amino acids, in this way blocking the accessibility of the proteolytic site. In addition, the pro-peptide covers conformational IgE epitopes of mature Der p 1, reducing the protein's allergenicity. Consequently, ProDerp1 is more hypoallergenic, which increases its safety as an immunotherapeutic for allergen-specific immunotherapy (AIT).

Recombinant ProDer p 1 has been successfully produced in the art in Pichia pastoris (see US2007/0122433), Drosophila melanogaster, mammalian cells and Escherichia coli, although E. coli produced aggregated ProDer p 1 derivatives. ProDer p 1 contains two N-glycosylation sites, one in the pro-peptide (asparagine34-lysine35-serine36 or N34-K35-S36) and one in the mature protein chain (N150-glutamine(Q)151-5152). Enzymatic deglycosylation of recombinant ProDer p 1 produced in P. pastoris has been shown to result in the spontaneous maturation (pro-peptide removal) of the allergen, suggesting that glycosylation may function as a shield covering the first maturation cleavage site.

In the present invention we have further improved the ProDerp1 allergen and found that deglycosylation, in particular N-glycan deglycosylation, particularly enzymatic N-glycan deglycosylation, leads to an improved hypoallergenic version of ProDerp1. Even more, when we additionally chemically inactivated the catalytic cysteine on position 132 of this deglycosylated form of ProDepr1 we obtained even a more improved hypoallergenic variant of ProDerp1.

Importantly, the invention is not limited to the specifically disclosed sequences of ProDerp1, but includes any hypoallergenic allergen which has its naturally occurring glycosylation groups (such as N-glycosylation) removed by enzymatic or chemical deglycosylation or has mutant glycosylation acceptor sites (such as N-glycosylation acceptor sites). Importantly such hypoallergenic allergens can be recognized by a decreased or abolished IgE-binding reactivity and/or histamine release activity, whilst retaining its T cell reactivity and/or the ability to stimulate an immune response against the wild-type allergen. The allergenic activity, and consequently the reduction in the allergenic activity, of these hypoallergenic allergens may be compared to the wild type by any of the following methods: histamine release activity or by IgE-binding reactivity, according to methods outlined in the materials and methods sections 22 and 23 described herein further.

In a first embodiment the invention provides a composition comprising at least two different allergens wherein said at least two allergens have a maximum of 20%, preferably a maximum of 10%, even more preferably a maximum of 5% of their natural glycans (such as for example N-glycans) as compared to the 100% glycans (such as for example N-glycans) present on said allergens in their natural environment wherein the maximum of 20%, preferably a maximum of 10%, even more preferably a maximum of 5% of their natural glycans (such as for example N-glycans) has been obtained by enzymatic deglycosylation of the at least 2 different allergens.

In yet another embodiment the invention provides a composition comprising at least two different allergens wherein said at least two allergens have between 5% and 20%, preferably between 5% and 10% of their natural glycans (such as for example N-glycans) as compared to the 100% glycans (such as for example N-glycans) present on said allergens in their natural environment.

In yet another embodiment the invention provides a composition comprising at least three different allergens wherein said at least three allergens have a maximum of 20% of their natural glycans (such as for example N-glycans) as compared to the 100% glycans (such as for example N-glycan) present on said allergens in their natural environment.

In yet another embodiment the invention provides a composition comprising at least three different allergens wherein said at least three allergens have between 5% and 20%, preferably between 5% and 10% of their natural glycans (such as for example N-glycans) as compared to the 100% glycans (such as for example N-glycans) present on said allergens in their natural environment.

The wording “have a maximum of 20% of their natural glycans (such as N-glycans) as compared to the 100% glycans (such as N-glycans)” refers to the fact that the allergen in its natural environment (e.g. an allergen derived from (or “obtained from” which is equivalent wording) a plant pollen) carries 100% of glycosylation groups (such as N-glycan groups). A chemical or enzymatic process to deglycosylate the glycans (such as N-glycans) on the allergen leads to a reduction of glycans (such as N-glycans) and this reduction is herein defined as to a remaining of maximum 20% of the glycans (such as N-glycans) with respect to the glycans (such as N-glycans) in their natural environment.

Enzymes to deglycosylate glycan structures (such as N-glycan structures) are known to the skilled glycobiologist and exemplified in the instant application for deglycosylation of N-glycans.

In yet another embodiment the invention provides a composition comprising at least two different allergens wherein said at least two allergens have a maximum of 20% of their natural N-glycans as compared to the 100% N-glycans present on said allergens in their natural environment wherein said allergens comprise non-functional N-glycan acceptor sites.

The wording “comprise non-functional N-glycan acceptor sites” refers to one or more, or all N-glycan acceptor sites which have been mutated (e.g. by recombinant engineering) to non-functional N-glycan acceptor sites and expressed in a suitable recombinant eukaryotic host.

In particular embodiments the allergens are derived from (or alternative wording: “are obtained from”) cockroaches, house dust mite, plant pollen, bee or wasp venom, domestic animals (cows, horses and the like) or pets (cats, dogs, guinea pigs and the like).

In yet another embodiment the invention provides a composition comprising an N-glycan deglycosylated, protease dead-modified proDerp1 protein which proDerp1 has been recombinantly made in a eukaryotic host, such as for example a lower eukaryotic host.

In a specific embodiment the invention provides a composition comprising an N-glycan deglycosylated, protease dead-modified proDerp1 protein produced in a recombinant eukaryotic host which ProDerp1 has been enzymatically deglycosylated with an enzyme with a specificity for N-glycans.

In a specific embodiment the invention provides a composition comprising a non-N-glycosylated, protease dead-modified proDerp1 protein which has been obtained via recombinant expression of a proDerp1 protein having non-functional N-glycan acceptor sites.

In yet another embodiment the invention provides a composition comprising a non-N-glycosylated, protease dead-modified proDerp1 protein which has no detectable cysteine protease activity.

In yet another embodiment the invention provides a composition comprising a non-N-glycosylated, protease dead-modified proDerp1 protein which has no detectable cysteine protease activity and which catalytic cysteine on position 132 in ProDerp1 has been alkylated on the thiol group.

Alkylation of the thiol groups of catalytic cysteines is common practice to kill the activity of cysteine proteases. Several agents for alkylation of thiol groups of cysteines are known in the art and include iodoacetamide, iodoacetic acid and the like.

In yet another embodiment the invention provides a composition comprising an enzymatically N-deglycosylated, protease dead-modified proDerp1 protein which has no detectable cysteine protease activity and which protein has been alkylated on the thiol group of the catalytic cysteine on position 132 in ProDerp1.

In yet another embodiment the invention provides a composition comprising an enzymatically N-deglycosylated, protease dead-modified proDerp1 protein which has no detectable cysteine protease activity and which protein has been alkylated on the thiol group of the catalytic cysteine on position 132 in ProDerp1 and wherein said protein has been obtained by production in the yeast Pichia pastoris.

In yet another embodiment the invention provides a composition comprising a ProDerp1 protein which is deglycosylated with an enzyme with a specificity for N-glycans and said protein has a thiol-alkylated cysteine on position 132 in SEQ ID NO: 1.

In yet another embodiment the invention provides a composition comprising a ProDerp1 protein which is deglycosylated with an enzyme with a specificity for N-glycans and said protein has a thiol-alkylated cysteine on position 132 in SEQ ID NO: 1 and which ProDerp1 has been produced in the yeast Pichia pastoris.

In yet another embodiment the invention provides a ProDerp1 protein, which sequence is depicted in SEQ ID NO: 1, obtained by recombinant production in the yeast Pichia pastoris which protein has been iodoalkylated after the production followed by deglycosylation with an enzyme with a specificity for N-glycans.

In yet another embodiment the invention provides a composition comprising an enzymatically deglycosylated, protease dead-modified proDerp1 protein which has no detectable cysteine protease activity and which has an amino acid substitution in the catalytic cysteine residue on position 132 of the amino acid sequence of ProDerp1.

In yet another embodiment the invention provides a composition obtained by the following steps: i) alkylation of the thiol group of cysteine on position 132 of recombinant proDerp1 which sequence is depicted in SEQ ID NO: 1 followed by ii) enzymatic deglycosylation of the thio-alkylated product with an enzyme with a specificity for N-glycans.

In yet another embodiment the invention provides a composition obtained by the following steps: i) enzymatic deglycosylation of recombinant ProDerp1 with an enzyme with a specificity for N-glycans, followed by alkylation of the thiol group of cysteine on position 132 of the obtained deglycosylated proDerp1 product.

In yet another embodiment the invention provides a composition comprising a non-N-glycosylated, protease dead-modified proDerp1 protein which has no detectable cysteine protease activity and which has an amino acid substitution in the catalytic cysteine residue on position 132 from cysteine to alanine in the amino acid sequence of ProDerp1.

In yet another embodiment the invention provides pharmaceutical compositions comprising a composition as described in one of the embodiments before and a pharmaceutical excipient.

In particular embodiments the hypoallergenic allergens of the invention have a substantially reduced allergenic activity.

“Substantially reduced allergenic activity” means that the allergenic activity as measured by residual IgE-binding activity is reduced to a maximum of 50% of the activity of the native unmodified or unmutated allergen, preferably to a maximum of 20%, more preferably to a maximum of 10%, still more preferably to a maximum of 5%, still more preferably to less than 5%. Alternatively, “substantially” also means that the histamine release activity of the mutant or variant is reduced by at least a 100-fold factor as compared to the native protein, preferably by a factor of 1000-fold, still more preferably by a factor of 10000-fold.

The immunogenicity of the mutant or variant allergen may be compared to that of the wild-type allergen by various immunological assays. The cross-reactivity of the mutant or variant and wild-type allergens may be assayed by in vitro T-cell assays after vaccination with either mutant or wild-type allergens. Briefly, splenic T-cells isolated from vaccinated animals may be restimulated in vitro with either mutant or wild-type allergen followed by measurement of cytokine production with commercially available ELISA assays, or proliferation of allergen specific T cells may be assayed over time by incorporation of tritiated thymidine. Also the immunogenicity may be determined by ELISA assay, the details of which may be easily determined by the man skilled in the art. Briefly, two types of ELISA assay are envisaged. First, to assess the recognition of the variant or mutant ProDerP1 by sera of mice immunized with the wild type ProDerP1; and secondly by recognition of wild type proDerP1 allergen by the sera of animals immunised with the mutant or variant ProDerp1 allergen.

The ProDerp1 products are recovered by conventional methods according to the host cell. Thus, where the host cell is a lower eukaryotic cell, the product may generally be isolated from the nutrient medium or from cell free extracts. Conventional protein isolation techniques include selective precipitation, absorption chromatography, and affinity chromatography including a monoclonal antibody affinity column.

In yet another embodiment the invention provides pharmaceutical, immunogenic and vaccine compositions comprising a hypoallergenic ProDerP1 derivative according to the invention, don-optimised or not, are also provided

In particular embodiments the pharmaceutical compositions of the present invention may include adjuvant compounds, or other substances which may serve to increase the immune response induced by the protein. The vaccine composition of the invention comprises an immunoprotective amount of the mutated or variant version of the ProDerP1 hypoallergenic protein. The term “immunoprotective” refers to the amount necessary to elicit an immune response against a subsequent challenge such that allergic disease is averted or mitigated. In the vaccine of the invention, an aqueous solution of the protein can be used directly. Alternatively, the protein, with or without prior lyophilization, can be mixed, adsorbed, or covalently linked with any of the various known adjuvants. Suitable adjuvants are commercially available such as, for example Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, and chemokines may also be used as adjuvants.

In the formulations of the invention it is preferred that the adjuvant composition induces an immune response predominantly of the Th1 type. High levels of Th1-type cytokines (e.g. IFN-gamma, TNFalpha, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. Accordingly, suitable adjuvants for use in eliciting a predominantly Th1-type response include, for example a combination of monophosphoryl lipid A, preferably 3-de-O-acylated monophosphoryl lipid A (3D-MPL) together with an aluminium salt. Other known adjuvants, which preferentially induce a Th1 type immune response, include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by Sato et al., Science 273:352, 1996. CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol. Another preferred adjuvant is a saponin, preferably QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210. A particularly potent adjuvant formulation involving QS21 3D-MPL & tocopherol in an oil in water emulsion is described in WO 95/17210 and is a preferred formulation. Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Accordingly there is provided an immunogenic composition comprising a ProDerP1 hypoallergenic variant or mutant as disclosed herein and an adjuvant, wherein the adjuvant comprises one or more of 3D-MPL, QS21, a CpG oligonucleotide, a polyethylene ether or ester or a combination of two or more of these adjuvants. The ProDerP1 hypoallergenic variant or mutant within the immunogenic composition is preferably presented in an oil in water or a water in oil emulsion vehicle.

The amount of the allergen of the present invention present in each vaccine dose is selected as an amount which induces an immunoprotective response without significant, adverse side effects in typical vaccines. Such amount will vary depending upon which specific allergen is employed and whether or not the vaccine is adjuvanted. Generally, it is expected that each dose will comprise 1-1000 μg of protein, preferably 1-200 μg. An optimal amount for a particular vaccine can be ascertained by standard studies involving observation of antibody titres and other responses in subjects. The vaccines of the present invention may be administered to adults or infants, however, it is preferable to vaccinate individuals soon after birth before the establishment of substantial Th2-type memory responses. Following an initial vaccination, subjects will preferably receive a boost in about 4 weeks, followed by repeated boosts every six months for as long as a risk of allergic responses exists.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a process for the production of a vaccine, comprising the steps of purifying a ProDerP1 variant or mutant according to the invention or a derivative thereof, by the process disclosed herein and admixing the resulting protein with a suitable adjuvant, diluent or other pharmaceutically acceptable excipient.

The present invention also provides a method for producing a vaccine formulation comprising mixing an allergen composition of the present invention together with a pharmaceutically acceptable excipient.

Another aspect of the invention is the use of a protein as claimed herein before for the manufacture of a vaccine for immunotherapeutically treating a patient susceptible to or suffering from allergy. A method of treating patients susceptible to or suffering from allergy comprising administering to said patients a pharmaceutically active amount of the immunogenic composition disclosed herein is also contemplated by the present invention.

A further aspect of the invention provides a method of preventing or mitigating an allergic disease in man (such as for example house dust mite allergy), which method comprises administering to a subject in need thereof an immunogenically effective amount of a mutated or variant allergen of the invention, or of a vaccine in accordance with the invention.

Therefore, the present invention includes pharmaceutical compositions that are comprised of a pharmaceutically acceptable carrier and a pharmaceutically effective amount of allergens or nucleotide sequences encoding said allergens and a pharmaceutically acceptable carrier. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of polypeptides and nucleotide sequences of the invention and a pharmaceutically acceptable carrier is preferably that amount which produces a result or exerts an influence on the particular allergenic condition being treated. The polypeptides and nucleotide sequences of the invention and a pharmaceutically acceptable carrier can be administered with pharmaceutically acceptable carriers well known in the art using any effective conventional dosage form, including immediate, slow and timed release preparations, and can be administered by any suitable route such as any of those commonly known to those of ordinary skill in the art. For therapy, the pharmaceutical composition of the invention can be administered to a patient in accordance with standard techniques. The administration can be by any appropriate mode, including orally, parenterally, topically, nasally, ophthalmically, sublingually, rectally, vaginally, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician.

The pharmaceutical composition of this invention can be lyophilized for storage and reconstituted in a suitable carrier prior to use.

When prepared as lyophilization or liquid, physiologically acceptable carrier, excipient, stabilizer need to be added into the pharmaceutical composition of the invention (Remington's Pharmaceutical Sciences 22th edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN™, PLURONICS™ or PEG and the like.

The preparation containing pharmaceutical composition of this invention should be sterilized before injection. This procedure can be done using sterile filtration membranes before or after lyophilization and reconstitution.

The pharmaceutical composition is usually filled in a container with sterile access port, such as an i.v. solution bottle with a cork. The cork can be penetrated by hypodermic needle.

It is to be understood that although particular embodiments, specific configurations as well as materials and/or molecules, have been discussed herein for nucleotide sequences, cells, polypeptides, and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

1. Prophylactic Treatment with IAA-Modified ProDer p 1 and ProDer p 1 C132A Glycoforms Reduces Eosinophilia in a HDM-Induced Asthma Mouse Model

In this therapeutic experiment it was investigated whether different N-glycans on IAA-modified ProDer p 1 and ProDer p 1 C132A forms were able to induce tolerance in a HDM-induced asthma model. The recombinant production of different glycoforms of ProDerp1 in Pichia pastoris is described in the materials and methods (section 20). The iodoalkylation (IAA) of ProDerp1 is also outlined in the materials and methods (section 18). The ProDerp1C132A form contains a mutation in the catalytic cysteine on position 132 (C→A mutation). In addition to the engineered allergen forms, IAA-modified DG ProDer p 1 (DG=deglycosylated) was included, which was generated by PNGase F treatment of Man₅GlcNAc₂ ProDer p 1. The IAA-modified ProDer p 1 and ProDer p 1 C132A forms used for this experiment were first analyzed on a Coomassie-stained SDS-PAGE gel

The patent or application file contains at least one drawing executed in color. Copies of this patent or patent application publication with color drawing(s) will be provided by the Office upon request arid payment of the necessary fee.

The drawings described are only schematic and are non-limiting. In the drawings, the size of some of the elements may be exaggerated and not drawn on scale for illustrative purposes.

A) to confirm their stability after freeze-thaw procedures. IAA-modified DG ProDer p 1 is mainly present as a single band corresponding to the theoretic molecular weight of ProDer p 1 (=34 kDa). In addition, the N-glycosylation profile of the various forms was analyzed with CE-LIF

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

B), confirming the absence of Man₅GlcNAc₂ residues on IAA-modified DG ProDer p 1

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

B, panel 4).

To initiate the therapeutic experiment, naïve C57BL/6J mice were prophylactically treated intranasally (i.n.) with 50 μg of an allergen form on days-14, -11, -7 and -1

The patent or application file contains at least one drawing executed in color. Copies of this patent or patent application publication with color drawing(s) will be provided by the Office upon request arid payment of the necessary fee.

The drawings described are only schematic and are non-limiting. In the drawings, the size of some of the elements may be exaggerated and not drawn on scale for illustrative purposes.

C). Subsequently, mice were sensitized intratracheally (i.t.) with 1 μg of HDM extract on day 0, and challenged i.n. with 10 μg of HDM extract on days 7-11. Asthma severity triggered by these challenges was measured by the quantification of immune cells in the BAL fluid by means of flow cytometry.

In general, we observed approximately similar reduced pulmonary inflammation when mice were pre-treated with either of the N-glycan variants of IAA-modified ProDer p 1 or ProDer p 1 C132A

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

D). By comparing IAA-modified Man₅GlcNAc₂ ProDer p 1 and Man₅GlcNAc₂ ProDer p 1 C132A, we could not observe any influence of the method used for inactivation of the catalytic cysteine on the degree of inflammation. However, to our surprise pulmonary inflammation was profoundly reduced when mice were pre-treated with IAA-modified DG ProDer p 1 (see materials and methods section 6). In particular, in this latter treatment the low number of infiltrating eosinophils, neutrophils, B cells and dendritic cells is remarkable compared to the other treatment groups. In addition, a very limited data spread was observed for the IAA-modified DG ProDer p 1-treated mice compared to the other treatment groups. A significant reduction in eosinophilia was initially observed for pre-treatment with LDN ProDer p 1 C132A but this finding could not be reproduced in a repeat experiment. However, the strong tolerizing effect of IAA-modified DG ProDer p 1-treatment was confirmed in the repeat experiment, with again a remarkably low number of infiltrating eosinophils, neutrophils, B cells and dendritic cells in the BAL fluid of challenged mice.

We conclude that a reduction in pulmonary inflammation was induced by intranasal pre-treatment with all engineered forms, but in particular PNGase F-deglycosylated (DG) IAA-modified ProDer p 1 treatment reduced the number of eosinophils, neutrophils, B cells and dendritic cells profoundly. Surprisingly and in contrast to current literature, we could not observe any consistent difference in tolerance induction between treatment with oligo-mannose, GalNAc₃GlcNAc₃Man₃GlcNAc₂ or GalNAc-β1,4-GlcNAc (LDN) N-glycan modified protein. Importantly, we could not confirm the induction of a regulatory immune phenotype by MGL-targeting as was previously described by Li and colleagues (Li D. et al (2012) J. Exp. Med. 209, 109-121).

2. Influence of the Removal of N-Linked Glycosylation of ProDerp1 and proDerp1C132A on the Therapeutic Efficacy

The in vivo experiments outlined in example 1 demonstrated that all different N-glycosylated variants of iodoacetic acid (IAA)-modified ProDer p 1 and ProDer p 1 C132A were able to reduce pulmonary eosinophilia in a HDM-driven asthma mouse model to a similar level. Surprisingly, prophylactic treatment with enzymatically deglycosylated (DG) IAA-modified ProDer p 1 reduced this eosinophilia even more, indicating an enhanced therapeutic effect in the absence of N-linked glycosylation. Therefore, in this example we focused on the production of ProDerp1 completely devoid of N-linked glycans. ProDer p 1 forms completely devoid of N-glycans were produced by genetically mutating both N-glycosylation sites (N34Q and N150Q), further referred to as the non-glycosylated (NG) forms. These NG forms carry no N-glycans. To investigate whether the approach of catalytic cysteine inactivation has an influence on the therapeutic potential, both NG IAA-modified ProDer p 1 N34Q/N150Q and ProDer p 1 C132A/N34Q/N150Q were included in further in vivo experiments, as well as the enzymatically deglycosylated (DG) ProDer p 1 C132A (containing some residual Man₅GlcNAc₂ N-glycans).

In this example, we either refer to deglycosylated (DG) ProDer p 1 variants where N-glycans are enzymatically removed, or non-glycosylated (NG) ProDer p 1 variants where N-glycans are removed by mutagenesis of the N-glycosylation acceptor site.

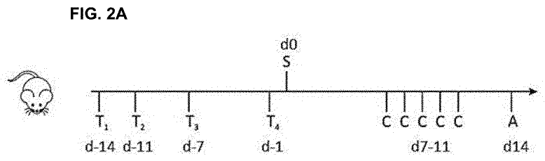

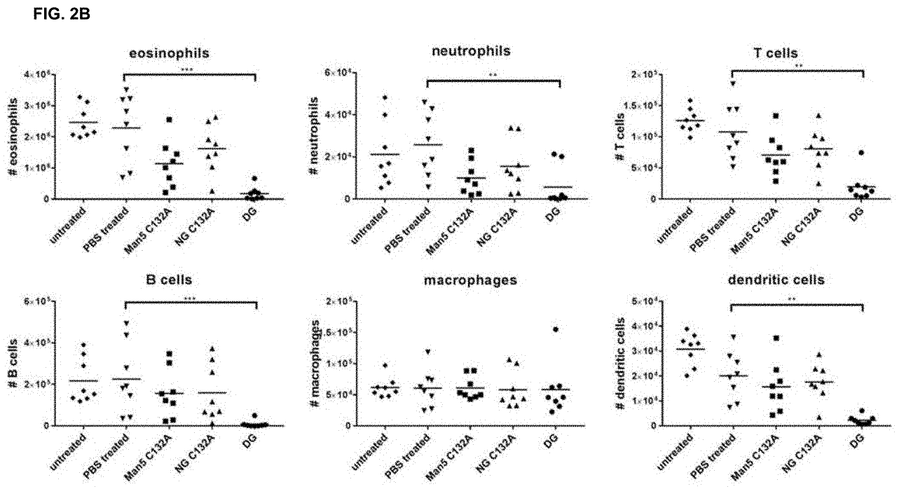

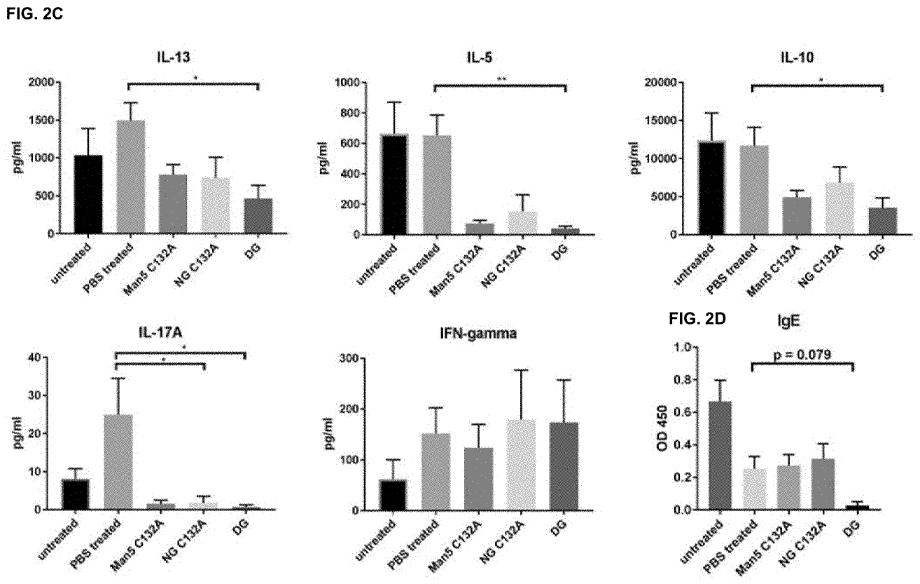

Besides IAA-modified DG ProDer p 1 and NG ProDer p 1 C132A/N34Q/N150Q, pre-treatment with Man₅GlcNAc₂ ProDer p 1 C132A was included to mimic the natural allergen glycan modification, and confirm the finding from example 1 of lower protection by N-glycan modification. Mice were pre-treated i.n. with 50 μg of a ProDer p 1 form, followed by sensitization and challenges according to the treatment scheme shown in FIG. 2A. On the day of analysis, mice were euthanized and bronchoalveolar lavage (BAL) fluid, lung draining mediastinal lymph nodes (MLNs) and blood were collected. The cellular composition of BAL fluid was analyzed and the protective effect of DG ProDer p 1, as described in example 1, was confirmed again, observed by a significant reduction in pulmonary eosinophilia, as well as a decreased number of B cells, dendritic cells, and T cells compared to the PBS-treated group (FIG. 2B). Pre-treatment with NG ProDer p 1 C132A/N34Q/N150Q (NG C132A) did also reduce pulmonary eosinophilia, although not as strong as pre-treatment with IAA-modified DG ProDer p 1. Pre-treatment with NG ProDer p 1 C132A/N34Q/N150Q gave similar results as pre-treatment with Man₅GlcNAc₂ProDer p 1 C132A. However, in a repeat of this experiment, pre-treatment with NG ProDer p 1 C132A/N34Q/N150Q did induce a statistically significant, protective effect, although again not as strong as IAA-modified DG ProDer p 1.

To evaluate T cell recall responses, lung-draining MLNs were isolated and cells were restimulated in vitro with HDM extract (15 μg/ml) to measure cytokine secretion (FIG. 2C). Type 2 cytokines IL-13 and IL-5 were significantly reduced in the IAA-modified DG ProDer p 1-treated group compared to the PBS-treated group. We could also see a trend in reduction of these cytokines in Man₅GlcNAc₂ ProDer p 1 C132A and NG ProDer p 1 C132A/N34Q/N150Q treated groups. A decrease in IL-10 and IL-17A production was observed for all ProDerp1 variants compared to the PBS and untreated mice. No significant differences could be observed in the secretion of IFN-γ between the various groups.

Furthermore, also blood samples were collected and analyzed for the presence of HDM-specific IgE. A noticeable reduction in HDM-specific IgE blood levels was observed for the IAA-modified DG ProDer p 1-treated group compared to the other groups (FIG. 2D). This result confirms reduced allergic asthma, considering IgE being a major mediator of the induction of allergic reactions.

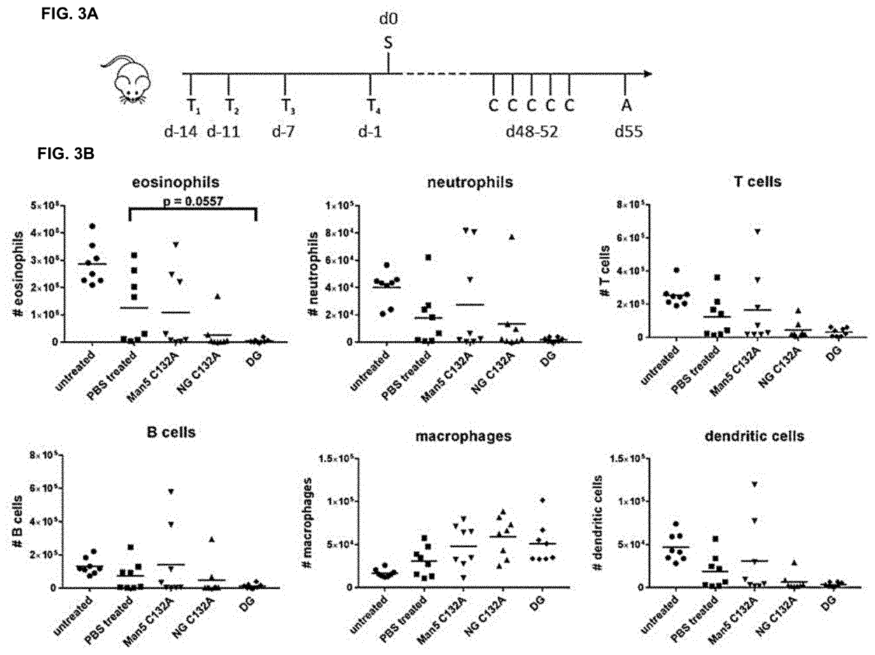

In a follow-up experiment, we investigated whether the protection induced by prophylactic treatment of mice was maintained over a long-term period. Therefore, mice were pre-treated i.n. with 50 μg of a ProDer p 1 form and sensitized according to the scheme shown in FIG. 3A. Challenge of mice was performed 7 weeks later. A lot of variation in the numbers of immune cells was observed in the PBS-treated group as well as in the Man₅GlcNAc₂ ProDer p 1 C132A-treated group (FIG. 3B). Although no significant differences compared to the PBS-treated group were obtained, pre-treatment with IAA-modified DG ProDer p 1 again showed high protection with almost no variation. All immune cells, except for the number of macrophages, were severely reduced. Pre-treatment with NG ProDer p 1 C132A/N34Q/N150Q also resulted in a decrease of the number of immune cells although not as strong as IAA-modified DG ProDer p 1, but significantly stronger than Man₅GlcNAc₂ProDerp1 C132A treatment.

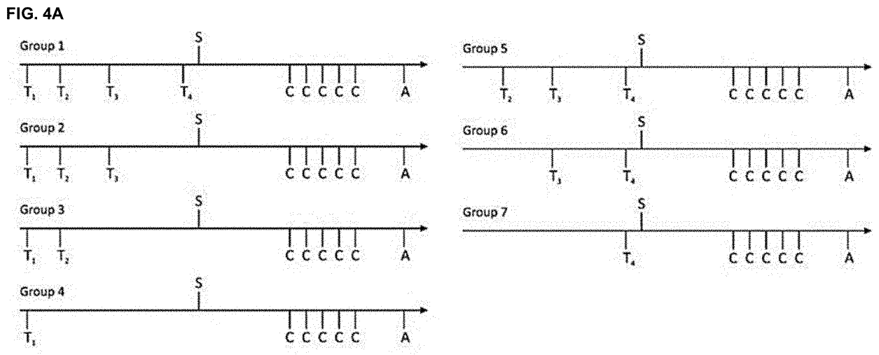

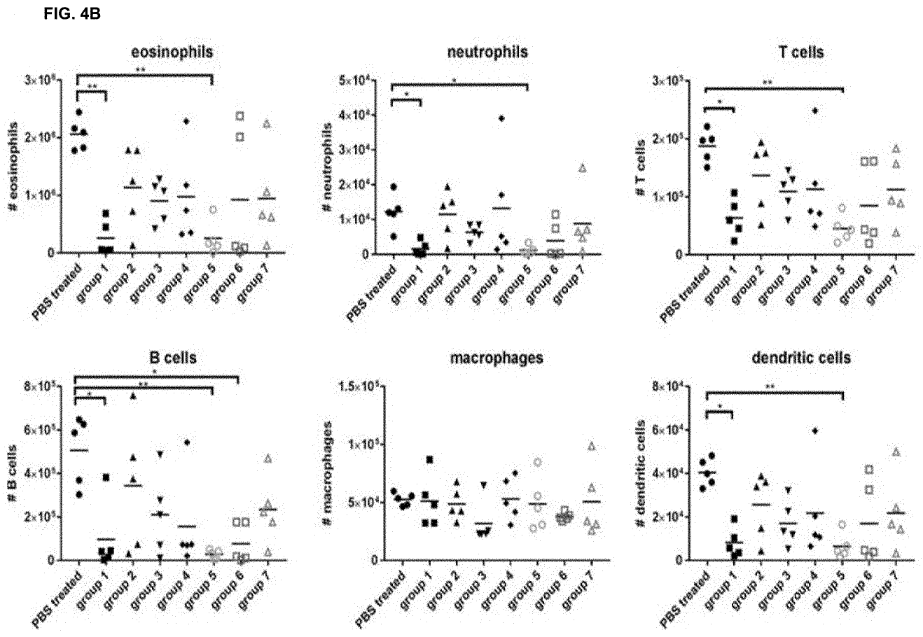

We conclude that we consistently showed a strong protective effect by pre-treatment with IAA-modified DG ProDer p 1 in the prevention of HDM-induced asthma in a mouse model. Therefore in a subsequent experiment, we investigated whether this protective effect was strong enough to reduce the number of treatments. Therefore, several pre-treatment schemes were designed as shown in FIG. 4A, followed by sensitization and challenge of mice to induce HDM-driven asthma. Treatment groups 1 and 5 showed a significant reduction in pulmonary inflammation compared to the PBS-treated group, suggesting that several treatments would be preferred to keep the protective effect, with the treatment administered the day before sensitization being preferred for tolerance induction (FIG. 4B).

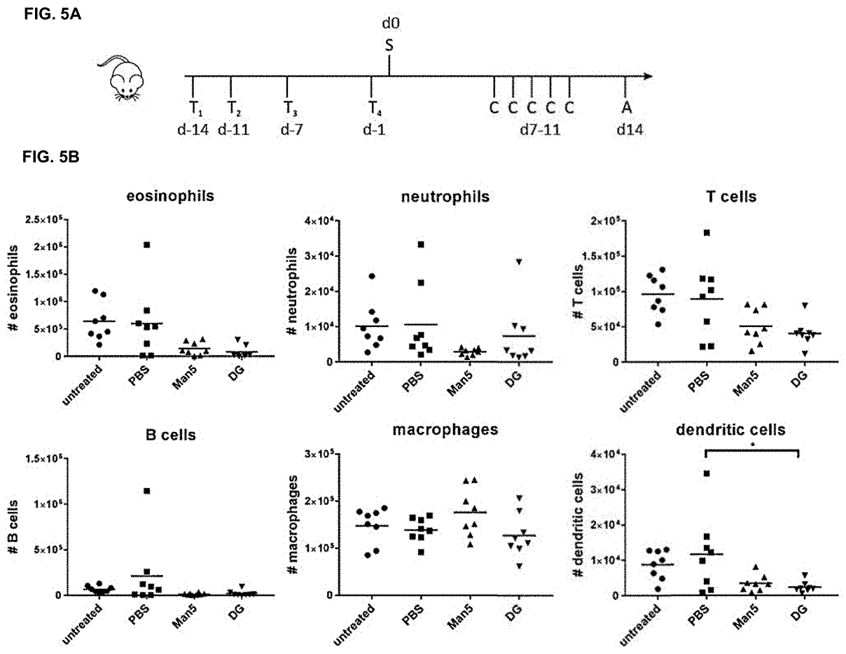

All previous experiments were performed in C57BL/6J mice, which made us wonder whether the observed increased protective effect of IAA-modified DG ProDer p 1 treatment was restricted to the HLA haplotype of C57BL/6J mice (haplotype b). Therefore, Balb/c mice, which carry a different HLA haplotype (haplotype d), were treated i.n. with 50 μg of either Man₅GlcNAc₂ or DG IAA-modified ProDer p 1, prior to sensitization and challenge of mice according to the scheme shown in FIG. 5A. As Balb/c mice have been described to be a prototypical Th2 strain, we expected these mice to be more susceptible to HDM-induced Th2-mediated allergic asthma. However, a lower eosinophil infiltration in the lung compared to C57BL6/J mice was observed for the PBS-treated control group. Pre-treatment with either ProDer p 1 form reduced pulmonary eosinophilia and the number of neutrophils, T cells, B cells and dendritic cells, to a similar level, compared to the PBS-treated group, although not statistically significant (FIG. 5B). The protection shows that the observed protective properties of ProDer p 1 treatment is not restricted to one HLA haplotype.

3. Antigen Uptake of Various ProDerp1 (C132A) Forms by Macrophages and DCs in the BAL Fluid

To obtain more insights in the mechanism of the protective effect induced by IAA-modified DG ProDerp1, we investigated whether the different ProDerp1 forms are taken up in a different way by macrophages and dendritic cells in the airways. Therefore, the purified Man₅GlcNAc₂-modified, DG and NG forms of both IAA-modified ProDer p 1 and ProDer p 1 C132A were labeled with AlexaFluor® 488. A similar labeling efficiency was obtained for each form so that we could directly compare the efficiency of antigen uptake. Cells obtained from BAL fluid of naïve C57BL/6J mice were stimulated ex vivo with 20 μg/ml of a labeled ProDer p 1 (C132A) form and harvested 0, 3 and 6 hours post stimulus. Antigen uptake by macrophages and dendritic cells was measured using flow cytometry.

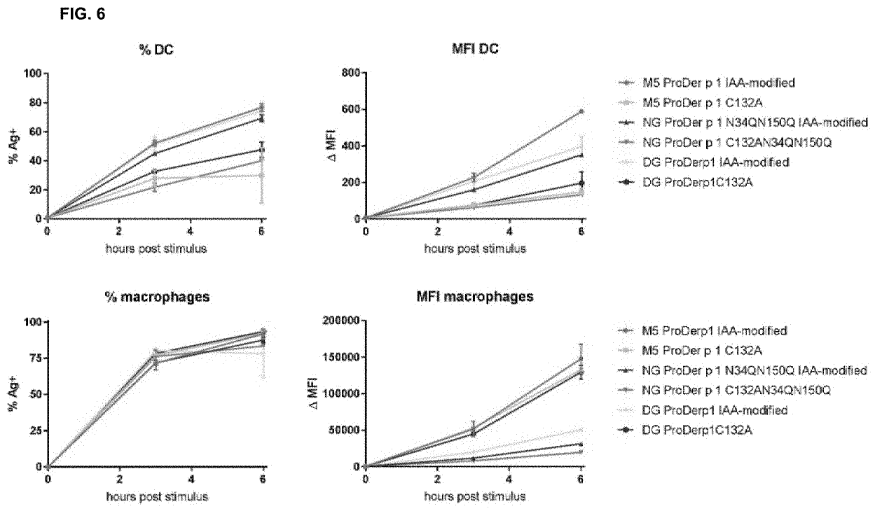

Antigen uptake by dendritic cells was remarkably higher for all three IAA-modified ProDer p 1 forms (shades of green) compared to the ProDer p 1 C132A forms (shades of red), with the highest antigen uptake observed for IAA-modified Man₅GlcNAc₂ ProDer p 1 (FIG. 6). Macrophages showed a higher uptake for Man₅GlcNAc₂-modified antigens. A higher antigen uptake could also be observed for DG ProDer p 1 C132A, likely because of the presence of remaining Man₅GlcNAc₂ N-glycan residues. This clear preference for the uptake of Man₅GlcNAc₂-modified antigens is probably because of the expression of the mannose receptor, assisting in binding and endocytosis of terminal mannose-modified molecules. From these results, we could conclude that the increased uptake of glycosylated forms by macrophages leads to the capture of these forms before efficient uptake and presentation by dendritic cells could occur. However, this should be further investigated in vivo. In combination with the increased uptake of the IAA-modified forms by dendritic cells, this may in part explain the stronger protective effect of the IAA-modified DG ProDer p 1 form. However, results from different experiments demonstrate that the IAA-modified NG ProDer p 1 N34Q/N150Q could not induce a protective effect as strong as the DG form, suggesting that an additional factor may be involved.

4. Characterization of Different Recombinant Protease Dead, Unglycosylated Forms of proDerp1

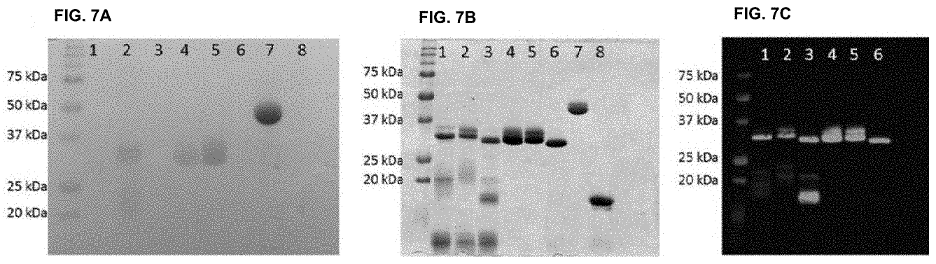

Different recombinant proDerp1 forms were recombinantly produced as described in the materials and methods section 4, purification was carried out as described in section 7. FIG. 7B shows Coomassie staining of iodoalkylated forms of enzymatically deglycosylated ProDerp1, Man₅GlcNAc₂-ProDerp1 and N-glycosylation mutants of ProDerp1 and C132A mutated forms of ProDerp1, Man₅GlcNAc₂-ProDerp1 and N-glycosylation mutants of ProDerp1. Remarkably in FIG. 7B, lane 1, there is a significant proteolytic degradation observed after PNGase F treatment of the IAA-modified ProDerp1 recombinant form while such proteolytic degradation is completely absent for the C132A variants (see lanes 4, 5 and 6 in FIG. 7B). In addition, a clear proteolytic degradation is found for the IAA-modified double N-glycosylation acceptor site mutant of ProDerp1 (see FIG. 7B, lane 3) but this degradation is different from the enzymatically deglycosylated IAA-modified form of ProDerp1 (see FIG. 7B, lane 1). Thus we observed the occurrence of a different type of clipping event (or degradation event) between the different IAA-modified ProDer p 1 forms, taking place either during production or purification. We refer to this clipping event as a type 1 clipping event for Man₅GlcNAc₂ IAA-modified ProDer p 1 and deglycosylated IAA-modified ProDer p 1, while the genetically non-glycosylated IAA-modified ProDer p 1 is clipped in a different way, further referred to as a type 2 clipping event. No clipping event occurs for the ProDer p 1 C132A variants. Although not wishing to limit the invention to a particular mechanism this difference may result in the exposure of different epitopes, contributing to the tolerance induction. Further research is necessary to determine the exact sites at which the hypoallergen is clipped. Initial molecular dynamics simulations performed for IAA-modified ProDer p 1 compared to ProDer p 1 C132A predicted the induction of a conformational change in or more flexibility of the loop between C201 and C215 (Martin Frank, Biognos). This change is predicted to be induced upon a conformational change occurring at the iodoalkylated cysteine of the IAA-modified ProDer p 1 variant. The conformational change in the loop may result in more exposure of this loop and a subsequent clipping event.

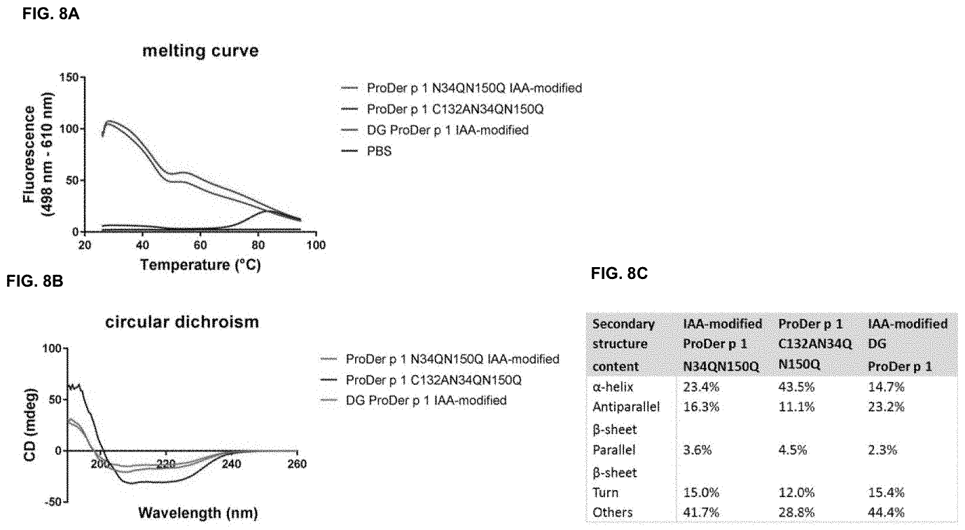

In a next step also the thermal stability of IAA-modified DG ProDer p 1, IAA-modified NG ProDer p 1 N34Q/N150Q and NG ProDer p 1 C132A/N34Q/N150Q was determined with a thermofluor assay. The NG ProDer p 1 C132A mutant exhibited a melting curve characterized by low initial fluorescence followed by as sigmoidal curve indicative of a protein unfolding transition (see FIG. 8A). The melting temperature was estimated to be around 75° C., which was similar as for Man₅GlcNAc₂ ProDer p 1 C132A. IAA-modified DG and NG ProDer p 1 displayed high initial fluorescence indicating the exposure of hydrophobic patches, possibly because of partial unfolding of the protein by IAA treatment. A second unfolding transition could be observed around 50-55° C. (FIG. 8A), similar to IAA-modified Man₅GlcNAc₂ ProDer p 1.

Additional information on the secondary structure content of the IAA-modified DG ProDer p 1, IAA-modified NG ProDer p 1 N34Q/N150Q and NG ProDer p 1 C132A/N34Q/N150Q variants was obtained by circular dichroism spectroscopy (see FIG. 8B). CD spectra of both IAA-modified DG and NG ProDer p 1 forms were similar with a considerable lower amount of α-helices and more irregular or unfolded regions compared to NG ProDer p 1 C132A (predicted with BeStSel, bestsel.elte.hu, 200-250 nm wavelength range) (see FIG. 8C).

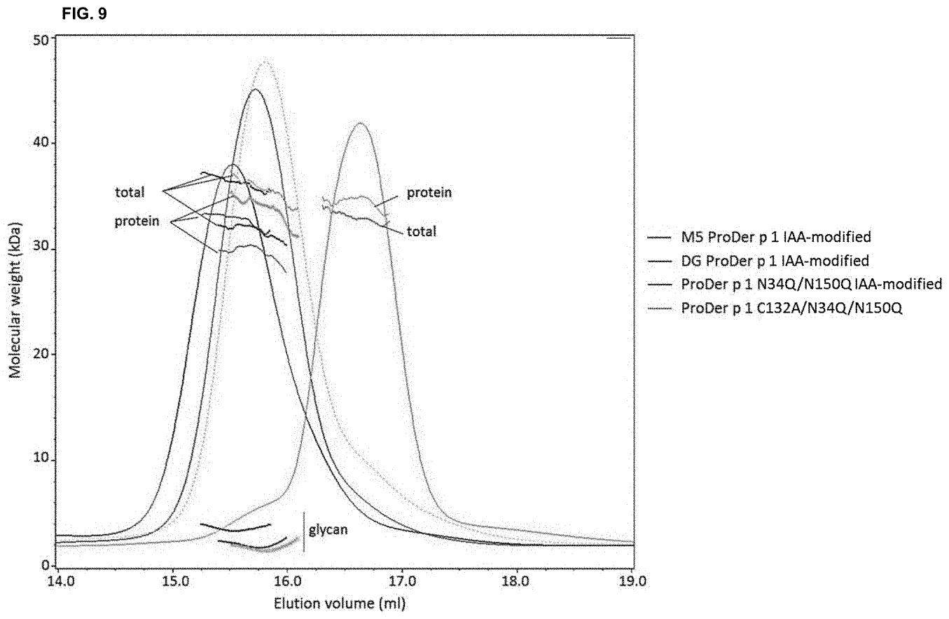

A difference in protein folding is also suggested from the data obtained with Size-Exclusion Chromatography-Multi Angle Laser Light Scattering (SEC-MALLS) (see FIG. 9). SEC-MALLS experimental data are further outlined in the materials and method section no. 23. The SEC profiles of IAA-modified Man₅GlcNAc₂ ProDer p 1, IAA-modified DG ProDer p 1 and IAA-modified NG ProDer p 1 N34Q/N150Q largely overlapped, while the SEC profile of NG ProDer p 1 C132A/N34Q/N150Q was shifted to the right, indicating a longer elution time. As the molecular weight of NG ProDer p 1 C132A/N34Q/N150Q was estimated to be similar as the molecular weight of the IAA-treated DG or NG ProDer p 1 forms, we interpret this as a smaller hydrodynamic volume of NG ProDer p 1 C132A/N34Q/N150Q due to a more compact protein fold.

All purified ProDer p 1 variants were in a monomeric state as their molecular weights were estimated between 30 and 40 kDa. Glycoprotein conjugate analysis of the SEC-MALLS data clearly showed the difference in glycan content between IAA-modified Man₅GlcNAc₂ ProDer p 1, and IAA-modified DG ProDer p 1 and NG ProDer p 1 N34Q/N150Q, which corresponds to the theoretic protein glycan modification. No glycans are present in NG ProDer p 1 C132A/N34Q/N150Q and this form is therefore not present in the figure. Thus a clear difference between glycosylated and de- or non-glycosylated forms was observed.

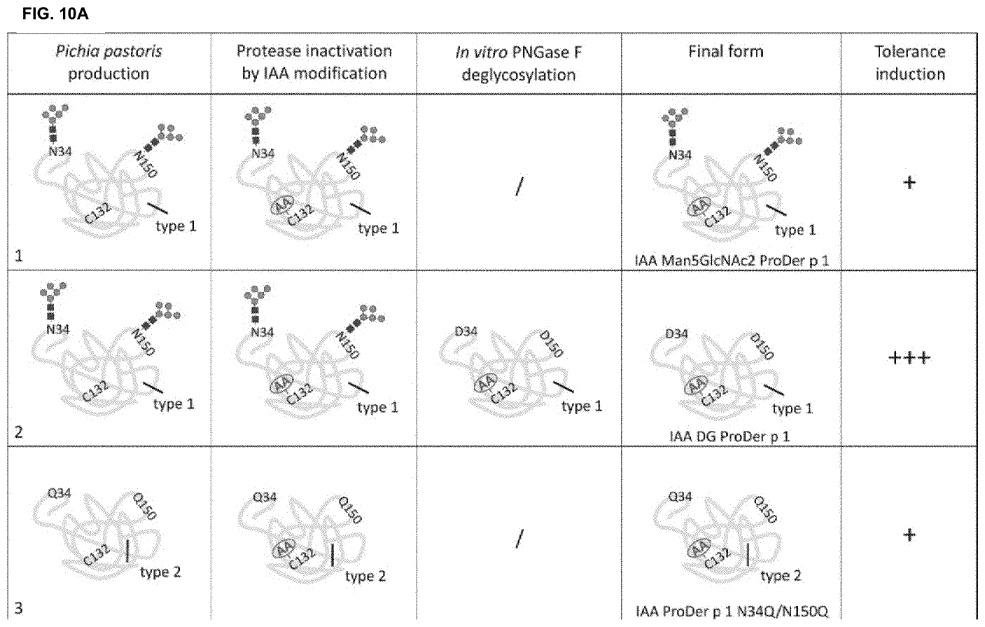

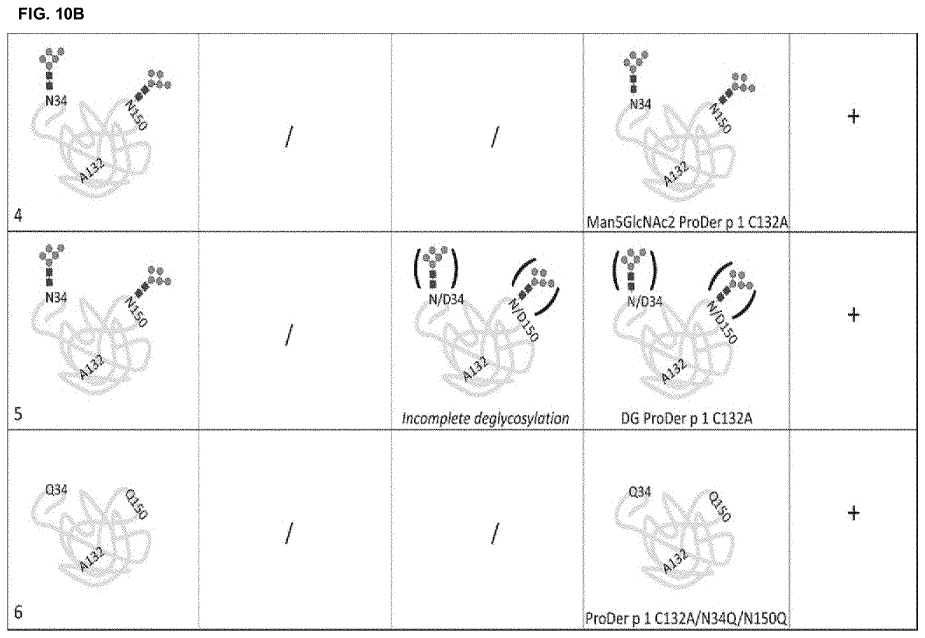

FIG. 10 depicts a schematic overview of the different ProDerp1 variants generated in the present invention.

5. Deglycosylation of Grass Pollen Extract

As described in the previous examples, prophylactic treatment with deglycosylated iodoalkylated ProDer p 1 results in the induction of tolerance in a HDM-driven allergic asthma model. Additionally, we aim to analyze whether a similar protective effect can be obtained for other allergens using a similar deglycosylation strategy. Plant allergens, such as grass pollen and ragweed pollen, are another major cause of allergic reactions and allergic asthma triggers. Most of these allergies are not caused by a predominant single allergen but by a complex mixture of several allergens, making recombinant allergen production cost-ineffective. Therefore, we deglycosylate a plant allergen extract, more specifically a Timothy grass pollen extract, that contains a complex mixture of plant allergens in order to determine whether deglycosylation of the naturally glycosylated allergens improves tolerance induction during allergen-specific immunotherapy.

To obtain effective deglycosylation of the grass pollen extract, PNGase A, PNGase F-II or PNGase H+ is used instead of PNGase F. These PNGases differ from PNGase F in substrate specificity and are able to cleave N-linked glycans with an α-1,3-fucose linked to the chitobiose core, while PNGase F is not. This immunogenic core α-1,3-fucose is often present on plant allergens, potentially eliciting hypersensitivity reactions in humans. Deglycosylation of the grass pollen extract is performed in non-denatured conditions in a buffer suitable for the particular PNGase. Successful deglycosylation is analyzed by comparing the CE-LIF profiles of PNGase-treated (in denatured conditions) grass pollen extract before and after the deglycosylation step. In addition, proteins are analyzed using SDS-PAGE and mass spectrometry to check for molecular weight shifts corresponding to the removal of N-glycans. To analyze whether the deglycosylated grass pollen extract is hypoallergenic and enhances tolerance induction compared to the natural grass pollen extract, a grass pollen-driven allergy murine model is used as described by Hesse and Nawijn (Hesse L. and Nawijn M. C. (2017) Methods Mol. Biol. 1559:137-168).

Materials and Methods

1. Construction of ProDer p 1 and ProDer p 1 C132A Expression Plasmids

The ProDer p 1 coding sequence was kindly provided by the VIB-UGent Protein Service Facility. The sequence was amplified using 5′-GTATCTCTCGAGAAAAGAGAGG (SEQ ID NO:2) forward (containing XhoI site, underlined) and 5′-GCGGCCGCGATTAGAGAATGACAACATATGG (SEQ ID NO:3) reverse (containing NotI site, underlined) primers with terminal XhoI/NotI restriction sites for cloning into the pPIC9 expression backbone (Invitrogen), generating the pPIC9ProDerp1 plasmid. The coding sequence was cloned in-frame with the α-mating factor prepro-sequence of Saccharomyces cerevisiae for secretion, under control of the strong methanol-inducible AOX1 promoter. The pPIC9 expression backbone carries the HIS4 gene for selection in his4 P. pastoris strains. Site-directed mutagenesis (QuickChange II Site-Directed Mutagenesis Kit, Agilent) of pPIC9ProDerp1 was performed using 5′-GGAGGCTGTGGTTCAGCTTGGGCTTTCTCTG (SEQ ID NO:4) forward and 5′-CAGAGAAAGCCCAAGCTGAACCACAGCCTCC (SEQ ID NO:5) reverse primers to induce a point mutation of the cysteine residue at position 132 to an alanine residue (C132A) (mutated codon underlined in the primers). The protocol was performed according to the manufacturer's instructions. The nucleotide sequence of both pPIC9ProDerp1 and pPIC9ProDerp1C132A was verified by Sanger sequencing at the VIB Genomics Core using 5′AOX1 and 3′AOX1 primers.

2. Transformation of P. Pastoris with Expression Plasmids

Transformation of P. pastoris was initially performed using the lithium acetate method as described by Lin-Cereghino J. et al (2005) Biotechniques 38, 44-48.

To ensure targeted genome integration, the expression plasmid was first linearized in the AOX1 promoter region using PmeI and was subsequently purified using Nucleospin® Gel and PCR Clean-up (Macherey-Nagel) to minimize salt concentration. Transformants were plated on CSM-HIS plates, which were supplemented with blasticidine for the maintenance of the GlycoSwitchM5® strain. Forty-eight single clones were picked to create a master-plate, used for subsequent clone screening.

3. Small-Scale Expression Screening of P. Pastoris Single Clones

Single clones were inoculated in 2 ml of BMGY in a 24-well plate sealed with an AirPore Tape Sheet (Qiagen), and incubated at 28° C. for 48 h, shaking. Cultures were centrifuged (3,000×g, 10 min, 4° C.), supernatant was removed and cell pellets were resuspended in 2 ml of BMMY. To induce protein expression, 1% (v/v) of methanol was added every 12 h during 48 h of incubation at 28° C. The cultures were centrifuged (3,000×g, 10 min, 4° C.) and supernatant was collected and stored at −20° C. until further use.

4. Expression Analysis of Proteins Secreted by P. Pastoris Strains

To analyze protein expression levels, proteins in 1 ml of culture medium, harvested from a small-scale screening, were precipitated using DOC/TCA. Briefly, 10% (v/v) of sodium deoxycholate (DOC, 5 mg/ml) was added to the samples followed by a 10-minute incubation on ice. Subsequently, 10% (v/v) of trichloroacetic acid (TCA) was added and samples were incubated on ice for 20 minutes. The samples were centrifuged (18,000×g, 30 min, 4° C.), supernatant removed and pellets were washed twice with 100% ice-cold acetone and once with 70% ethanol. Pellets were resuspended in D-PBS (Lonza) and equal volumes were analyzed with sodium dodecyl sulfate polyacrylamide gel electrophoresis (SDS-PAGE) using 12% Tris-glycine gels. Proteins were either visualized by Coomassie Brilliant Blue-staining or by western blot. For western blot, the proteins were blotted on a nitrocellulose membrane using a Pierce™ Power Blotter (Thermo Fischer Scientific) and the membrane was subsequently blocked with 5% (w/v) of milk powder in PBS containing 0.05% Tween-20 (PBST). ProDer p 1 was visualized using rabbit anti-Der p 1 IgG polyclonal antibody (LS-C149183, LifeSpan BioSciences, 0.5 μg/ml, 5% (w/v) of milk powder in PBST), followed by a second incubation with goat anti-rabbit DyLight 800 conjugated IgG antibody (SA5-35571, Thermo Scientific, 67 ng/ml, 5% (w/v) of milk powder in PBST). Proteins were visualized using a LI-COR® Odyssey Detection System (Westburg).

5. N-Glycan Analysis Using Capillary Electrophoresis-Laser-Induced Fluorescence (CE-LIF)

The N-glycosylation profile of glycoproteins was analyzed by capillary electrophoresis with laser-induced fluorescence detection and was performed as described by Laroy et al.²¹ Briefly, either secreted proteins in 500 μl of culture medium obtained from a small-scale screening experiment or 10 μg of purified protein were denatured in 8 M urea, 360 mM Tris pH 8.6, 3.2 mM EDTA and subsequently blotted on a PVDF membrane. Disulfide bridges were reduced using 0.1 M dithiothreitol and blocked by carboxymethylation using 0.1 M iodoacetic acid (IAA) to avoid reformation of the disulfide bonds. The PVDF membrane was blocked with 1% polyvinylpyrrolidone 360 and the N-glycans were released from the bound proteins using 0.9 IUBMB mU of peptide-N-glycosidase from Flavobacterium meningosepticum (PNGase F, recombinantly in-house produced from E. coli as a His-tagged protein, 9 IUBMB mU/μl) in 10 mM Tris-acetate buffer pH 8.3. These N-glycans were subsequently labeled with the fluorescent dye 8-aminopyrene-1,3,6-trisulfonic acid (APTS; 1:1 mix of 20 mM APTS in 1.2 M citric acid with 0.5 M 2-picoline borane in DMSO) for detection. The excess of APTS was removed from the labeled samples using a 96-well Sephadex G10 post-derivatization clean-up step and samples were resuspended in 10 μl of ultrapure water. N-glycan samples were diluted 10 times in ultrapure water for the subsequent detection on a multi-capillary ABI 3130 DNA sequencer according to the settings described by Laroy et al.²¹ To determine the composition of the N-glycans, sequential exoglycosidase digests (Table 1) were performed on the APTS-labeled N-glycans (1 μl of APTS-labeled N-glycans, 0.2 μl of the appropriate amount of enzyme, 0.1 μl of 50 mM ammonium acetate pH 5.0 and 0.7 μl of ultrapure water, overnight incubation at 37° C.). After overnight digestion, the reaction volumes were adapted to 15 μl with ultrapure water and samples were analyzed using the ABI 3130 DNA sequencer.

TABLE 1
Exoglycosidases.

| Exoglycosidase | Specificity |
|---|---|
| α-1,2-mannosidase (T. reesei; in-house production) | α-1,2-mannose |
| Mannosidase (Jack bean; in-house dialyzed) | α-1,2/3/6-mannose |

6. PNGase F Digest

PNGase F digests were performed, both on P. pastoris culture medium, as well as on purified proteins. For the digest on culture medium, 1 ml of ice-cold 100% acetone was added to 500 μl of medium and incubated on ice for 20 minutes. Proteins were precipitated by centrifugation (18,000×g, 2 min, 4° C.), the supernatant was removed and the pellet was dried. Subsequently, proteins were denatured by the addition of 12.5 μl of 10× glycoprotein denaturing buffer (5% SDS, 0.4 M DTT) and 100 μl of 50 mM Tris-acetate pH 8.3, followed by a 5-minute incubation at 95-100° C. After denaturation, an overnight PNGase F digest was performed by the addition of 1.5 μl of 10% NP-40, 1.25 μl of 10× G7 buffer (500 mM sodium phosphate pH 7.5), 5 μl of 25× complete EDTA-free protease inhibitor (Roche), 1 μl of PNGase F (in-house production, 9 IUBMB mU/μl) and purified water to a final volume of 125 μl. The next day, proteins were precipitated using the previously described DOC/TCA protocol and analyzed with SDS-PAGE.

For PNGase F digests performed on purified proteins, 1 μg (in case of protein visualization using western blot) or 3 μg (in case of Coomassie Brilliant Blue-staining) of protein was denatured by a 10-minute incubation step at 100° C. in a total volume of 10 μl containing 1 μl of 10× glycoprotein denaturing buffer (5% SDS, 0.4 M DTT). Subsequently, 2 μl of 10× G7-buffer (500 mM sodium phosphate pH 7.5), 2 μl of 10% NP-40 and 1 μl of PNGase F (in-house production, 9 IUBMB mU/μl) were added and the volume was adjusted to 20 μl with purified water. Samples were incubated for 1 hour at 37° C. and analyzed with SDS-PAGE.

7. Purification of ProDer p 1 and ProDer p 1 C132A from P. Pastoris Culture Medium

A pre-culture of 5 ml BMGY containing 100 μg/ml blasticidine was inoculated with a clone of the ProDer p 1- or ProDer p 1 C132A-expressing GlycoSwitchM5® strain and incubated overnight at 28° C., shaking. This pre-culture was used to inoculate 1 L of BMGY (4×250 ml in 2 L baffled shake flasks). After 48 h of cell culture growth in BMGY medium and another 48 h of methanol-induced protein expression in BMMY medium, the supernatant was collected and used for purification on an ÄKTA Protein Purification System (GE Healthcare). Prior to purification, supernatant harvested from the ProDer p 1-expressing GlycoSwitchM5® strain was incubated with 10 mM IAA (in the dark, RT, 30 min) to block any remaining cysteine protease activity. As a capture step, hydrophobic interaction chromatography (HIC) was performed. Hereto, 1.5 M (NH₄)₂SO₄was added to the supernatant, which was subsequently loaded onto a pre-equilibrated (50 mM Tris-HCl pH 7.4+1.5 M (NH₄)₂SO₄) HiTrap Phenyl FF HS column (5 ml, GE Healthcare). Proteins were eluted by reducing the salt concentration using a stepwise gradient of 30% and 100% of 50 mM Tris-HCl pH 7.4 elution buffer. Protein containing fractions were pooled and desalted on a SephadexG25 gel filtration column (XK26/40 column, GE Healthcare, pre-equilibrated with 25 mM Tris-HCl pH 7.8). Protein containing fractions were loaded onto a pre-equilibrated HiScreen Q FF column (GE Healthcare, 25 mM Tris-HCl pH 7.8) for anion exchange chromatography (AEX). Elution of bound proteins was performed by increasing salt concentrations using a stepwise gradient of 10%, 30%, 50% and 100% of 25 mM Tris-HCl pH 7.8+1 M NaCl elution buffer. A final polishing step was performed using size-exclusion chromatography (SEC) on a Superdex 75 10/300 GL column (GE Healthcare), pre-equilibrated with PBS. Protein concentrations were measured with the Eppendorf BioSpectrometer® (A280, extinction coefficient: 52,175 M⁻¹cm⁻¹), and purified proteins were stored at −80° C.

8. Circular Dichroism Spectroscopy

To rapidly evaluate the secondary structure content of the protein forms, circular dichroism (CD) measurements were performed. Therefore, protein samples were buffer exchanged to 10 mM potassium phosphate pH 7.6, using Amicon Ultra-0.5 Centrifugal Filter Unit with Ultracel-10 membrane (Merck) according to the manufacturer's instructions. CD spectra were obtained for Man₅GlcNAc₂ProDer p 1 and Man₅GlcNAc₂ ProDer p 1 C132A, measured at 200 μg/ml on a J-710 spectropolarimeter (Jasco) with a scan speed of 50 nm/min at RT. Spectra were recorded as the average of 9 scans between 190 and 260 nm.

9. Thermofluor Assay

To analyze thermal stability of the protein forms, a thermofluor assay was performed. A dilution series of the protein of interest was made ranging from 50 μl of 20 ng/μl to 225 ng/μl. 3.13 μl of a 300× working solution of SYPRO™ Orange (5000× concentrate in DMSO, Life Technologies, S-6650) was added to the protein samples. Samples were divided in triplicates in a qPCR plate (Lightcycler® 480 Multiwell Plate 96, Roche) and run on a Lightcycler® 480 (Roche) according to settings shown in Table 2.

TABLE 2
Settings Lightcycler ® 480 thermal shift assay.

| Temperature | | Temperature ramp |
|---|---|---|
| (° C.) | Acquisition | (° C./s) |
| 25 | No | 4.4 |
| 95 | Continuous | 0.02 |
| 25 | No | 2.2 |
| Filter combination | Excitation 498 nm | Emission: 610 nm |

10. Mass Spectrometry Analysis of Intact Proteins

1 μl (10 pmol) of protein material was analyzed by LC-MS/MS on a Ultimate 3000 split flow HPLC (Thermo Fisher Scientific, Bremen, Germany) in-line connected with an ESI source to a Q Exactive HF mass spectrometer (Thermo Fischer Scientific). The proteins were separated through a size exclusion column (made-in-house, 1.0 mm I.D.×50 mm, 5 μm beads Reprosil 50 SEC, Dr. Maisch) using an isocratic gradient of 30/70 ACN/H2O at a flow rate of 10 μl/min for 5 min. The mass spectrometer was operated in MS1 mode at a resolution of 120 000, a SID of 40 V, a spray voltage of 3.8 kV, capillary temperature of 320° C., a sheath gas of 10, 3 microscans, an AGC target of 3E6, a maximum iontime of 200 ms and a mass range from 1000-3000 m/z in profile mode.

11. In Vivo 1-Der Proliferation

1-Der CD4+ T cells were isolated from spleens and lymph nodes of 1-Der mice (Plantinga M et al. (2013) Immunity 38, 322-335), and labeled with CFSE (Invitrogen) in PBS. 3.3.10⁶ cells were injected intravenously (i.v.) in the tail of naïve C57BL/6J mice on day 0. On day 1, these mice were sedated with isoflurane (2.5-3% isoflurane in air) and treated intratracheally (i.t.) with 1 μg/70 μl PBS of a ProDer p 1 (C132A) form. Treatments included IAA-modified Man₅GlcNAc₂, GlcNAc₃Man₃GlcNAc₂ and GalNAc₃GlcNAc₃Man₃GlcNAc₂ ProDer p 1, as well as LDN ProDer p 1 C132A. As a positive control, HDM extract (10 μg in 70 μl of PBS) was included, and as negative controls, mice were instilled with PBS and PBS+endoT (20 ng of endoT in 70 μl PBS), as residual endoT could be detected in the glyco-engineered forms. On day 4, mice were euthanized by an overdose of pentobarbital (300 mg/kg body weight) intraperitoneally (i.p.) and mediastinal lymph nodes (MLNs) were isolated. Cell suspensions were obtained by homogenization through a 70 μM cell strainer, cells were counted and stained with Live/Dead Fixable Aqua stain (Invitrogen), anti-CD16/32 (2.4G2, Fc block, BD Biosciences), anti-CD4-PerCP (RM4-5, BD Biosciences), anti-CD3-APC (17A2, BD Biosciences), anti-V134 TCR-PE (KT4, BD Biosciences), anti-CD69-V450 (H1.2F3, BD Biosciences), anti-CD44-BV605 (IM7, BD Biosciences) for 30 minutes at 4° C. in PBS supplemented with 2 mM EDTA and 0.5% BSA. Measurements were performed on a BD LSRFortessa cytometer (BD Biosciences) and data were analyzed using FlowJo LLC). The division index, proliferation index and median fluorescence intensity of CFSE signal were determined based on the Vβ4+CFSE+ cell population. The division index represent the average number of cell divisions that a cell in the 1-Der T cell population has undergone, while the proliferation index excludes the undivided peak by representing the total number of divisions by the number of cells that went into division.

12. Prophylactic Treatment in a House Dust Mite-Induced Asthma Mouse Model

For all manipulations, mice were sedated with isoflurane (2.5-3% isoflurane in air). On days-14, -11, -7 and -1, naïve C57BL/6J mice were pretreated (intranasal (i.n.) instillation) with 50 μg of a ProDer p 1 (C132A) form in 40 μl of PBS. An untreated control group, in which mice were sedated without subsequent treatment, and a PBS-treated group were included. On day 0, mice were i.t. sensitized with 1 μg of HDM extract and on days 7-11 mice were daily challenged i.n. with 10 μg of HDM extract. On day 14, mice were euthanized by an overdose of pentobarbital i.p. (300 mg/kg body weight) and bronchoalveolar lavage (BAL) was performed using 3×1 ml of PBS containing 2 mM EDTA. BAL fluid was centrifuged (400×g, 5 min, 4° C.) and resuspended in 300 μl of PBS supplemented with 2 mM EDTA and 0.5% BSA. Cells were stained with Fixable Viability Dye eFluor™ 780 (Thermo Fischer Scientific), anti-CD16/32 (2.4G2, Fc block, BD Biosciences), anti-SiglecF-PE (E50-2440, BD Biosciences), anti-CD19-PE-Cy7 (SJ25C1, BD Biosciences), anti-CD11b-BV605 (M1/70 BD Biosciences), anti-CD11c-APC (HL3, BD Biosciences), anti-Ly6G-AF700 (1A8, BD Biosciences), anti-MHClI-FITC (M5/114.15.2, Thermo Fischer Scientific) and anti-CD3-PE-Cy7 (17A2, BD Biosciences) for 30 minutes at 4° C. Absolute cell numbers were quantified by means of CountBright™ Absolute counting Beads (Thermo Fisher Scientific). Measurements were performed on a BD LSRFortessa cytometer (BD Biosciences) and data were analyzed using FlowJo (FlowJo, LLC).

13. Statistical Analyses

Statistics was performed making use of a non-parametric Kruska I-Wallis test with Dunn's test for multiple comparisons. Differences were considered significant when the p-value was lower than 0.05.

14. N-Glycosylation Site Mutants—Construct Design and Development

To remove the N-glycosylation sites N34 and N150 in the sequence of ProDer p 1 and ProDer p 1 C132A, site-directed mutagenesis (QuickChange II Site-Directed Mutagenesis Kit, Agilent) was performed to replace both amino acids by a Q according to the manufacturer's instructions. pPIC9ProDerp1 and pPIC9ProDerp1C132A were used as starting vectors and the used primer sets are shown in Table. The sequences of pPIC9ProDerp1N34Q, pPIC9ProDerp1N150Q, pPIC9ProDerp1N34Q/N150Q, pPIC9ProDerp1C132AN34Q, pPIC9ProDerp1C132AN150Q and pPIC9ProDerp1C132A/N34Q/N150Q were verified by Sanger sequencing at the VIB Genomics Core using 5′AOX1 and 3′AOX1 primers.

TABLE 3
Primer sets for site-directed mutagenesis to obtain N-glycosylation site mutants.

| Site of | | Primer | SEQ |
|---|---|---|---|
| mutagenesis | Primer set | purification | ID |
| N34Q | Fw: 5′-GAATACAAAAAAGCCTTCCAGAAAAGTTATGCTACCTTCG-3′ | HPLC | 6 |
| | Rev: 5′- CGAAGGTAGCATAACTTTTCTGGAAGGCTTTTTTGTATTC-3′ | | 7 |
| N150Q | Fw: 5′-GCTTATTTGGCTTACCGTCAGCAATCATTGGATCTTGC-3′ | HPLC | 8 |
| | Rev: 5′-GCAAGATCCAATGATTGCTGACGGTAAGCCAAATAAGC-3′ | | 9 |

Fw: forward primer;
Rev: reverse primer.

15. Protein Purification of ProDer p 1 and ProDer p 1 C132A N-Glycosylation Site Mutants

Protein purification of ProDer p 1 and ProDer p 1 C132A N-glycosylation site mutants was performed with a combination of HIC, a desalting step followed by AEX and a final SEC step, as described in Chapter 4. Protein concentrations were measured with the Eppendorf BioSpectrometer® (A280, extinction coefficient: 52,175 M⁻¹cm⁻¹), and purified proteins were stored in PBS at −80° C.

16. Expression of ProDer p 1 by Pichia Pastoris

Secretion of the hypoallergenic ProDer p 1 was obtained by a fusion of the protein sequence to the S. cerevisiae α-mating factor prepro-sequence, under the control of the strong methanol-inducible AOX1 promoter. Expression of ProDer p 1 by the GS115 (his4) P. pastoris strain resulted in the efficient secretion of ProDer p 1, detected as a diffuse band around 34 kDa to 50 kDa on a SDS-PAGE gel. After treatment with PNGase F, ProDer p 1 migrated as several distinct bands around 34 kDa and lower, which confirms N-linked hyper-glycosylation of ProDer p 1, and reveals the occurrence of protein maturation and/or degradation upon deglycosylation. Analysis of the N-glycans using CE-LIF, in which the N-glycans are separated according to their hydrodynamic volume and charge, the hyper-glycosylation could be identified as yeast-specific, high-mannose residues.

The first step towards N-glycosylation engineering involved the expression of ProDer p 1 in the GlycoSwitchM5® strain, which modifies glycoproteins mainly with Man₅GlcNAc₂ residues. A high level of protein secretion was obtained, mainly displayed as two bands migrating between 25 kDa and 37 kDa on SDS-PAGE, conform to the theoretical molecular weight of 34 kDa. Compared to the non-transformed GlycoSwitchM5® strain, an additional diffuse band of low intensity could be observed around 50 kDa, likely corresponding to residual high-mannose background. Indeed, analysis of the N-glycosylation profile mainly revealed the presence of Man₅GlcNAc₂ residues, and a low amount of remaining high-mannose peaks could be detected as well.

17. Purification Optimization of Man₅GlcNAc₂ ProDer p 1

As the GlycoSwitchM5® strain is the initiating strain for further glyco-engineering, purification of ProDer p 1 was optimized based on culture medium obtained from the ProDer p 1-expressing GlycoSwitchM5® strain. High level expression of Der p 1 by P. pastoris was previously obtained through the secretion of ProDer p 1 followed by a maturation step post-purification in an acidic buffer. Because of the induction of maturation at low pH, it is of utmost importance to avoid acidic buffers. Despite this effort to avoid protein maturation, we observed a lot of protein degradation during our first purification attempts, resulting in the removal of almost all intact protein. As ProDer p 1 is a protease, we assumed that the reduced enzymatic activity of ProDer p 1 may still be sufficient to induce degradation, perhaps in combination with auto-maturation. It has been demonstrated before that mature Der p 1 is able to activate other ProDer p 1 molecules. Therefore, we aimed to block the proteolytic activity of the enzyme by modifying, via iodo-alkylation, the catalytic cysteine residue on the one hand, or by genetically mutating this cysteine residue on the other hand.

18. Iodoacetic Acid Modification of the Catalytic Cysteine in ProDerp1 Variants and Mutants

The positive effect of IAA modification of the cysteine in the catalytic site could already be observed with SDS-PAGE when comparing the supernatant of the Man₅GlcNAc₂ ProDer p 1-expressing strain without and with IAA treatment. Almost no intact ProDer p 1 could be detected before cysteine modification, while IAA treatment increased the protein stability considerably. The IAA-treated culture medium was subsequently loaded on a Phenyl Sepharose column to capture our protein of interest based on HIC. The Man₅GlcNAc₂ ProDer p 1-containing fractions obtained after elution were pooled and desalted by gel filtration, which is required for a subsequent AEX step. Most of the Man₅GlcNAc₂ ProDer p 1 already eluted from the AEX Q Sepharose column at 100 mM and 300 mM salt concentration, and pooled fractions were polished using SEC (Superdex 75). Most of the hyperglycosylated ProDer p 1 background appeared to be removed by AEX. The purified sample migrated as two distinct bands with some residual degradation present. The yield obtained from a 1 L expression culture was around 70 mg.

A PNGase F digest was performed on the purified sample, confirming the presence of N-linked glycans on the protein. As the molecular weight of PNGase F (35.6 kDa) is similar to the molecular weight of ProDer p 1 (34 kDa), a Der p 1-targeted western blot was necessary to distinguish ProDer p 1 from PNGase F on a SDS-PAGE gel. The PNGase F-digested sample migrated as a single band on SDS-PAGE, which ran slightly lower than both bands present in the untreated sample. This suggests that the lower band of the untreated sample corresponded to ProDer p 1 modified with one N-glycan and the upper band to ProDer p 1 modified with two N-glycans. The distinct, single band on SDS-PAGE after the PNGase F digest, is also suggestive for the complete absence of O-linked glycosylation, which was confirmed by intact protein mass spectrometry. Although the main part of the purified protein appeared to be intact, some protein degradation could still be detected in the purified sample.

19. Site-Directed Mutagenesis of the Catalytic Cysteine

As an alternative to the cysteine iodoalkylation necessary for eliminating the proteolytic activity, the amino acid was genetically mutated to an alanine (C132A). Expression of ProDer p 1 C132A by both GS115 and GlycoSwitchM5® strains was similar to the expression of ProDer p 1, resulting in a diffuse band and two distinct single bands on SDS-PAGE, respectively. Analysis of the N-glycosylation profiles with CE-LIF confirmed the high-mannose modification by the GS115 strain, and the modification with mainly Man₅GlcNAc₂ residues by the GlycoSwitchM5® strain. Again, some residual high-mannose residues could be detected on the ProDer p 1 expressed by the GlycoSwitchM5® strain, which was observed as a light diffuse band on SDS-PAGE and corresponding to the low intensity peaks on the N-glycosylation profile. Man₅GlcNAc₂ ProDer p 1 C132A was purified using the same combination of HIC, desalting, AEX and SEC, but this time without prior IAA-treatment.

PNGase F treatment of the purified Man₅GlcNAc₂ ProDer p 1 C132A suggested again the partial occupation of both N-glycosylation sites, migrating on a SDS-PAGE as two distinct bands of which the lower and upper band correspond to single or double N-glycosylated ProDer p 1, respectively. No degradation bands could be detected, neither by visualization with Coomassie-staining nor by Der p 1-targeted western blot.

20. The Development of Glyco-Engineered ProDerp1 and ProDerp1 C132A Glycoforms in P. Pastoris

The first step to obtain glyco-engineered forms of ProDer p 1 is the expression of the hypoallergen in the GlycoSwitchM5® P. pastoris strain. The GlycoSwitchM5® strain is the initiating strain (Jacobs P. P. et al (2009) Nat. Protoc. 4, 58-70) for further glyco-engineering using the GlycoSwitch® technology, to achieve other glycoforms. In a wild-type yeast strain, folded Man₈GlcNAc₂-modified glycoproteins are transported from the ER to the Golgi apparatus for further extension with high-mannose residues. The initial step for this high-mannosylation is the addition of an α-1,6-mannose residue to the α-1,3-mannose residue of the trimannosyl-core by the Och1p. In the GlycoSwitchM5® strain, the Och1p α-1,6-mannosyltransferase locus has been engineered to largely eliminate the immunogenic and yeast-specific high-mannose N-glycans.

Thus, the ProDer p 1-expressing GlycoSwitchM5® strain was used as the initiating strain for further N-glycosylation engineering using the GlycoSwitch® technology. In between each engineering step, the N-glycosylation profile was analyzed with CE-LIF. Insertion of GnT-I in the Man₅GlcNAc₂ ProDerp 1-expressing strain generated an almost complete conversion of the N-glycans to GlcNAcMan₅GlcNAc₂ residues. Subsequent overexpression of Man-II resulted in the removal of the terminal α-1,3- and α-1,6-mannose residues, generating GlcNAcMan₃GlcNAc₂ N-glycans. Introduction of GnT-II restored strain stability, resulting in a quite homogenous modification of ProDer p 1 with GlcNAc₂Man₃GlcNAc₂. Further extension towards the tri-antennary GlcNAc₃Man₃GlcNAc₂ N-glycan was obtained after the introduction of GnT-IV. Prior to subsequent in vitro enzymatic GalNAc-transfer with the mutant human beta-1,4-galactosyltransferase (mutation Y285L), GlcNAc₃Man₃GlcNAc₂ ProDerp1 was treated with IAA and purified.

21. Determination of IgE-Binding Activity of the Allergens

Immunoplates are coated overnight with specific allergens (e.g. ProDerP1 variants as described herein) (500 ng/well) at 4° C. Plates are then washed 5 times with 100 μl per well of TBS-Tween buffer (50 mM Tris-HCl pH 7.5, 150 mm NaCl, 0.1% Tween 80) and saturated for 1 hr at 37° C. with 150 μl of the same buffer supplemented with 1% BSA. Sera from allergic patients (e.g. allergic to D. pteronyssinus) and diluted at 1/8 were then incubated for 1 hr at 37° C. Plates are washed 5 times with TBS-Tween buffer and the allergen-IgE complexes are detected after incubation with a mouse anti-human IgE antibody (Southern Biotechnology Associates) and a goat anti-mouse IgG antibody coupled to alkaline phosphatase (dilution 1/7500 in TBS-Tween buffer, Promega). The enzymatic activity is measured using the p-nitrophenylphosphate substrate (Sigma) dissolved in diethanolamine buffer (pH 9.8). OD.sub.410 nm was measured in a Biorad Novapath ELISA reader. For IgE inhibition assays, plates are coated with the allergen (such as ProDerP1 derivatives) at the same concentration (0.12 μM). A pool of 20 human sera from allergic patients (RAST value>100 kU/L) is preincubated overnight at 4° C. with various concentrations (3.6-0.002 μM) of allergen (such as a recombinant ProDerP1 variant) as inhibitors and added on ELISA plates. IgE-binding is detected as described above.

22. Histamine Release

The histamine release is assayed using leukocytes from the peripheral heparinized blood of an allergic donor and by the Histamine-ELISA kit (Immunotech). Basophils are incubated with serial dilutions of allergen (such as a recombinant ProDerP1 variant) for 30 min at 37° C. The total amount of histamine in basophils is quantified after cell disruption with the detergent IGEPAL CA-630 (Sigma).

23. Aggregation Propensity Studies Using Dynamic Light Scattering (DLS) and Size-Exclusion Chromatography Multi-Angle Laser Light Scattering (SEC-MALLS)

To analyze aggregation of the protein samples, 0.5 mg/ml of IAA-modified DG ProDer p 1 SEC-eluted fractions were centrifuged (18,000×g, 10 minutes, 4° C.) and 70 μl was analyzed on Zetasizer Nano-S (Malvern Instruments), using disposable cuvettes (UV-Cuvette micro 70 Brand).

For SEC-MALLS, 150 μl of 0.5 mg/ml protein samples were injected onto a Superdex 200 Increase 10/300 GL SEC column (GE Healthcare), with PBS as running buffer at 0.5 ml/min, coupled to an online SPD-10A UV-VIS detector (Shimadzu), a multi-angle laser light scattering miniDAWN TREOS instrument (Wyatt) and a Optilab T-rEX refraction index detector (Wyatt) at 25° C. As a calibration reference, bovine serum albumin (Albumin standard, Thermo Fisher Scientific) was used. For the molecular mass determination of the glycan modification, a refractive index increment (dn/dc) value of 0.160 mL/g was used. Data were recorded and analyzed using the ASTRA software package (Wyatt, v6.1).

1. A method of producing a composition, the method comprising:
alkylating a recombinant protein comprising SEQ ID NO: 1 (proDerp1) at the thiol group of the cysteine at position 132 of SEQ ID NO: 1; and
enzymatically deglycosylating the thio-alkylated recombinant protein with an enzyme specific for N-glycans.

2. The method according to claim 1, further comprising producing the recombinant protein in a recombinant fungal or yeast cell.

3-10. (canceled)

11. A composition comprising the alkylated and deglycosylated recombinant protein produced by the method of claim 1.

12. The composition of claim 11, wherein the composition is a pharmaceutical composition and where the composition further comprises a pharmaceutical excipient.

13. The composition of claim 11, wherein the composition further comprises an adjuvant.

14. The composition of claim 12, wherein the adjuvant is a Th1-inducing adjuvant.

15. A method of active immunotherapy, the method comprising administering to a mammalian subject the composition of claim 11.
